SEQUENCE LISTING (180 sequences)

SEQ ID NO: 1 - DNA, 37 bp, Artificial Sequence
  Description of Artificial Sequence Synthetic oligonucleotide
cagacaaagactaccctacgcgttcaagatggcctac

SEQ ID NO: 2 - DNA, 21 bp, Artificial Sequence
  Description of Artificial Sequence Synthetic oligonucleotide
gttaaatcgtggatagtagac

SEQ ID NO: 3 - DNA, 21 bp, Artificial Sequence
  Description of Artificial Sequence Synthetic oligonucleotide
caggtacatttgctcgtcctt

SEQ ID NO: 4 - DNA, 51 bp, Artificial Sequence
  Description of Artificial Sequence Synthetic oligonucleotide
aaggacgagcaaatgtacctgcagacaaagactaccctacgcgttcaagat

SEQ ID NO: 5 - DNA, 58 bp, Artificial Sequence
  Description of Artificial Sequence Synthetic oligonucleotide
gtaggccatcttgaacgcgtagggtagtctttgtctggtctactatccacgatttaac

SEQ ID NO: 6 - DNA, 21 bp, Artificial Sequence
  Description of Artificial Sequence Synthetic oligonucleotide
caggtacatttgctcgtcctt

SEQ ID NO: 7 - DNA, 21 bp, Artificial Sequence
  Description of Artificial Sequence Synthetic oligonucleotide
aaggacgagcaaatgtacctg

SEQ ID NO: 8 - DNA, 18 bp, Artificial Sequence
  Description of Artificial Sequence Synthetic oligonucleotide
ggaacagctttgaggtgt

SEQ ID NO: 9 - DNA, 18 bp, Artificial Sequence
  Description of Artificial Sequence Synthetic oligonucleotide
acacctcaaagctgttcc

SEQ ID NO: 10 - DNA, 21 bp, Artificial Sequence
  Description of Artificial Sequence Synthetic oligonucleotide
gtctactatccacgatttaac

SEQ ID NO: 11 - DNA, 21 bp, Artificial Sequence
  Description of Artificial Sequence Synthetic oligonucleotide
gttaaatcgtggatagtagac

SEQ ID NO: 12 - DNA, 26 bp, Artificial Sequence
  Description of Artificial Sequence Synthetic oligonucleotide
ggaacagctttgaggtgtgtgtttgt

SEQ ID NO: 13 - DNA, 28 bp, Artificial Sequence
  Description of Artificial Sequence Synthetic oligonucleotide
gttaaatcgtggatagtagacttcgcac

SEQ ID NO: 14 - DNA, 28 bp, Artificial Sequence
  Description of Artificial Sequence Synthetic oligonucleotide
gtgcgaacaggtacatttgctcgtcctt

SEQ ID NO: 15 - DNA, 41 bp, Artificial Sequence
  Description of Artificial Sequence Synthetic oligonucleotide
aaggacgagcaaatgtacctgcaggaacagctttgaggtgt

SEQ ID NO: 16 - DNA, 49 bp, Artificial Sequence
  Description of Artificial Sequence Synthetic oligonucleotide
acaaacacacacctcaaagctgttcctggtctactatccacgatttaac

SEQ ID NO: 17 - DNA, 30 bp, Artificial Sequence
  Description of Artificial Sequence Synthetic oligonucleotide
acggaacagctttgaggtgtgtgtttgtgc

SEQ ID NO: 18 - DNA, 30 bp, Artificial Sequence
  Description of Artificial Sequence Synthetic oligonucleotide
acggaacagctttgaggtgcgtgtttgtgc

SEQ ID NO: 19 - DNA, 43 bp, Artificial Sequence
  Description of Artificial Sequence Synthetic oligonucleotide
aaggacgagcaaatgtacctgcagtcaagatcacagattttgg

SEQ ID NO: 20 - DNA, 49 bp, Artificial Sequence
  Description of Artificial Sequence Synthetic oligonucleotide
gcccgcccaaaatctgtgatcttgactggtctactatccacgatttaac

SEQ ID NO: 21 - DNA, 30 bp, Artificial Sequence
  Description of Artificial Sequence Synthetic oligonucleotide
atgtcaagatcacagattttgggcgggcca

SEQ ID NO: 22 - DNA, 30 bp, Artificial Sequence
  Description of Artificial Sequence Synthetic oligonucleotide
atgtcaagatcacagattttgggctggcca

SEQ ID NO: 23 - DNA, 41 bp, Artificial Sequence
  Description of Artificial Sequence Synthetic oligonucleotide
aaggacgagcaaatgtacctgcaaggtcacgaggagtacag

SEQ ID NO: 24 - DNA, 49 bp, Artificial Sequence
  Description of Artificial Sequence Synthetic oligonucleotide
tcattgcactgtactcctcgtgaccttggtctactatccacgatttaac

SEQ ID NO: 25 - DNA, 30 bp, Artificial Sequence
  Description of Artificial Sequence Synthetic oligonucleotide
gcaggtcacgaggagtacagtgcaatgagg

SEQ ID NO: 26 - DNA, 30 bp, Artificial Sequence
  Description of Artificial Sequence Synthetic oligonucleotide
gcaggtcaagaggagtacagtgcaatgagg

SEQ ID NO: 27 - DNA, 30 bp, Artificial Sequence
  Description of Artificial Sequence Synthetic oligonucleotide
ataggtggttttggtctagctacagtgaaa

SEQ ID NO: 28 - DNA, 30 bp, Artificial Sequence
  Description of Artificial Sequence Synthetic oligonucleotide
ataggtgattttggtctagctacagtgaaa

SEQ ID NO: 29 - DNA, 41 bp, Artificial Sequence
  Description of Artificial Sequence Synthetic oligonucleotide
aaggacgagcaaatgtacctgcaaggtggttttggtctagc

SEQ ID NO: 30 - DNA, 50 bp, Artificial Sequence
  Description of Artificial Sequence Synthetic oligonucleotide
ttcactgtagctagaccaaaaccaccttggtctactatccacgatttaac

SEQ ID NO: 31 - DNA, 30 bp, Artificial Sequence
  Description of Artificial Sequence Synthetic oligonucleotide
ataggtgattttggtctagctacagagaaa

SEQ ID NO: 32 - DNA, 40 bp, Artificial Sequence
  Description of Artificial Sequence Synthetic oligonucleotide
aaggacgagcaaatgtacctgcaaggtgattttggtctag

SEQ ID NO: 33 - DNA, 49 bp, Artificial Sequence
  Description of Artificial Sequence Synthetic oligonucleotide
tctctgtagctagaccaaaatcaccttggtctactatccacgatttaac

SEQ ID NO: 34 - DNA, 30 bp, Artificial Sequence
  Description of Artificial Sequence Synthetic oligonucleotide
ttcaaaaagatcaaagtgctggcctccggt

SEQ ID NO: 35 - DNA, 30 bp, Artificial Sequence
  Description of Artificial Sequence Synthetic oligonucleotide
ttcaaaaagatcaaagtgctgggctccggt

SEQ ID NO: 36 - DNA, 43 bp, Artificial Sequence
  Description of Artificial Sequence Synthetic oligonucleotide
aaggacgagcaaatgtacctgcacaaaaagatcaaagtgctgg

SEQ ID NO: 37 - DNA, 49 bp, Artificial Sequence
  Description of Artificial Sequence Synthetic oligonucleotide
cggaggccagcactttgatctttttgtggtctactatccacgatttaac

SEQ ID NO: 38 - DNA, 30 bp, Artificial Sequence
  Description of Artificial Sequence Synthetic oligonucleotide
gcctacgtgatggccatcgtggacaacccc

SEQ ID NO: 39 - DNA, 30 bp, Artificial Sequence
  Description of Artificial Sequence Synthetic oligonucleotide
gcctacgtgatggccagcgtggacaacccc

SEQ ID NO: 40 - DNA, 49 bp, Artificial Sequence
  Description of Artificial Sequence Synthetic oligonucleotide
ggttgtccacgatggccatcacgtagtggtctactatccacgatttaac

SEQ ID NO: 41 - DNA, 30 bp, Artificial Sequence
  Description of Artificial Sequence Synthetic oligonucleotide
gtgcagctcatcatgcagctcatgcccttc

SEQ ID NO: 42 - DNA, 30 bp, Artificial Sequence
  Description of Artificial Sequence Synthetic oligonucleotide
gtgcagctcatcacgcagctcatgcccttc

SEQ ID NO: 43 - DNA, 42 bp, Artificial Sequence
  Description of Artificial Sequence Synthetic oligonucleotide
aaggacgagcaaatgtacctgcagcagctcatcatgcagctc

SEQ ID NO: 44 - DNA, 49 bp, Artificial Sequence
  Description of Artificial Sequence Synthetic oligonucleotide
agggcatgagctgcatgatgagctgctggtctactatccacgatttaac

SEQ ID NO: 45 - DNA, 30 bp, Artificial Sequence
  Description of Artificial Sequence Synthetic oligonucleotide
tggccaaacagctgggtgcggaagagaaag

SEQ ID NO: 46 - DNA, 30 bp, Artificial Sequence
  Description of Artificial Sequence Synthetic oligonucleotide
tggccaaactgctgggtgcggaagagaaag

SEQ ID NO: 47 - DNA, 41 bp, Artificial Sequence
  Description of Artificial Sequence Synthetic oligonucleotide
aaggacgagcaaatgtacctgcagccaaacagctgggtgcg

SEQ ID NO: 48 - DNA, 50 bp, Artificial Sequence
  Description of Artificial Sequence Synthetic oligonucleotide
tttctcttccgcacccagctgtttggctggtctactatccacgatttaac

SEQ ID NO: 49 - DNA, 30 bp, Artificial Sequence
  Description of Artificial Sequence Synthetic oligonucleotide
actacctttctacggacgtgggattctgca

SEQ ID NO: 50 - DNA, 30 bp, Artificial Sequence
  Description of Artificial Sequence Synthetic oligonucleotide
actacctttctacggacgtgggatcctgca

SEQ ID NO: 51 - DNA, 41 bp, Artificial Sequence
  Description of Artificial Sequence Synthetic oligonucleotide
aaggacgagcaaatgtacctgcatacctttctacggacgtg

SEQ ID NO: 52 - DNA, 49 bp, Artificial Sequence
  Description of Artificial Sequence Synthetic oligonucleotide
cagaatcccacgtccgtagaaaggtatggtctactatccacgatttaac

SEQ ID NO: 53 - DNA, 30 bp, Artificial Sequence
  Description of Artificial Sequence Synthetic oligonucleotide
ttccagtggccatcaaagtgtcgagggaaa

SEQ ID NO: 54 - DNA, 30 bp, Artificial Sequence
  Description of Artificial Sequence Synthetic oligonucleotide
ttccagtggccatcaaagtgttgagggaaa

SEQ ID NO: 55 - DNA, 41 bp, Artificial Sequence
  Description of Artificial Sequence Synthetic oligonucleotide
aaggacgagcaaatgtacctgcaccagtggccatcaaagtg

SEQ ID NO: 56 - DNA, 49 bp, Artificial Sequence
  Description of Artificial Sequence Synthetic oligonucleotide
tccctcgacactttgatggccactggtggtctactatccacgatttaac

SEQ ID NO: 57 - DNA, 30 bp, Artificial Sequence
  Description of Artificial Sequence Synthetic oligonucleotide
ggatgtgcggctcatacacagggacttggc

SEQ ID NO: 58 - DNA, 30 bp, Artificial Sequence
  Description of Artificial Sequence Synthetic oligonucleotide
ggatgtgcggctcgtacacagggacttggc

SEQ ID NO: 59 - DNA, 41 bp, Artificial Sequence
  Description of Artificial Sequence Synthetic oligonucleotide
aaggacgagcaaatgtacctgcaatgtgcggctcatacaca

SEQ ID NO: 60 - DNA, 49 bp, Artificial Sequence
  Description of Artificial Sequence Synthetic oligonucleotide
caagtccctgtgtatgagccgcacattggtctactatccacgatttaac

SEQ ID NO: 61 - DNA, 24 bp, Artificial Sequence
  Description of Artificial Sequence Synthetic oligonucleotide
cttgtggtagttggagctgctggc

SEQ ID NO: 62 - DNA, 24 bp, Artificial Sequence
  Description of Artificial Sequence Synthetic oligonucleotide
cttgtggtagttggagctggtggc

SEQ ID NO: 63 - DNA, 40 bp, Artificial Sequence
  Description of Artificial Sequence Synthetic oligonucleotide
aaggacgagcaaatgtacctgcaacttgtggtagttggag

SEQ ID NO: 64 - DNA, 48 bp, Artificial Sequence
  Description of Artificial Sequence Synthetic oligonucleotide
gccagcagctccaactaccacaagttggtctactatccacgatttaac

SEQ ID NO: 65 - DNA, 24 bp, Artificial Sequence
  Description of Artificial Sequence Synthetic oligonucleotide
cttgtggtagttggagcttgtggc

SEQ ID NO: 66 - DNA, 41 bp, Artificial Sequence
  Description of Artificial Sequence Synthetic oligonucleotide
aaggacgagcaaatgtacctgcaacttgtggtagttggagc

SEQ ID NO: 67 - DNA, 48 bp, Artificial Sequence
  Description of Artificial Sequence Synthetic oligonucleotide
gccacaagctccaactaccacaagttggtctactatccacgatttaac

SEQ ID NO: 68 - DNA, 24 bp, Artificial Sequence
  Description of Artificial Sequence Synthetic oligonucleotide
cttgtggtagttggagctgatggc

SEQ ID NO: 69 - DNA, 48 bp, Artificial Sequence
  Description of Artificial Sequence Synthetic oligonucleotide
gccatcagctccaactaccacaagttggtctactatccacgatttaac

SEQ ID NO: 70 - DNA, 24 bp, Artificial Sequence
  Description of Artificial Sequence Synthetic oligonucleotide
cttgtggtagttggagctcgtggc

SEQ ID NO: 71 - DNA, 48 bp, Artificial Sequence
  Description of Artificial Sequence Synthetic oligonucleotide
gccacgagctccaactaccacaagttggtctactatccacgatttaac

SEQ ID NO: 72 - DNA, 24 bp, Artificial Sequence
  Description of Artificial Sequence Synthetic oligonucleotide
cttgtggtagttggagctagtggc

SEQ ID NO: 73 - DNA, 48 bp, Artificial Sequence
  Description of Artificial Sequence Synthetic oligonucleotide
gccactagctccaactaccacaagttggtctactatccacgatttaac

SEQ ID NO: 74 - DNA, 24 bp, Artificial Sequence
  Description of Artificial Sequence Synthetic oligonucleotide
cttgtggtagttggagctgttggc

SEQ ID NO: 75 - DNA, 48 bp, Artificial Sequence
  Description of Artificial Sequence Synthetic oligonucleotide
gccaacagctccaactaccacaagttggtctactatccacgatttaac

SEQ ID NO: 76 - DNA, 24 bp, Artificial Sequence
  Description of Artificial Sequence Synthetic oligonucleotide
cttgtggtagttggagctggttgc

SEQ ID NO: 77 - DNA, 48 bp, Artificial Sequence
  Description of Artificial Sequence Synthetic oligonucleotide
gcaaccagctccaactaccacaagttggtctactatccacgatttaac

SEQ ID NO: 78 - DNA, 30 bp, Artificial Sequence
  Description of Artificial Sequence Synthetic oligonucleotide
cttgtggtagttggagctggtgacgtaggc

SEQ ID NO: 79 - DNA, 30 bp, Artificial Sequence
  Description of Artificial Sequence Synthetic oligonucleotide
cttgtggtagttggagctggtggcgtaggc

SEQ ID NO: 80 - DNA, 41 bp, Artificial Sequence
  Description of Artificial Sequence Synthetic oligonucleotide
aaggacgagcaaatgtacctgcatgtggtagttggagctgg

SEQ ID NO: 81 - DNA, 49 bp, Artificial Sequence
  Description of Artificial Sequence Synthetic oligonucleotide
ctacgtcaccagctccaactaccacatggtctactatccacgatttaac

SEQ ID NO: 82 - DNA, 30 bp, Artificial Sequence
  Description of Artificial Sequence Synthetic oligonucleotide
cttgtggtagttggagctggtgtcgtaggc

SEQ ID NO: 83 - DNA, 49 bp, Artificial Sequence
  Description of Artificial Sequence Synthetic oligonucleotide
ctacgacaccagctccaactaccacatggtctactatccacgatttaac

SEQ ID NO: 84 - DNA, 30 bp, Artificial Sequence
  Description of Artificial Sequence Synthetic oligonucleotide
acccagaatcagaaggtgggagaactgaag

SEQ ID NO: 85 - DNA, 30 bp, Artificial Sequence
  Description of Artificial Sequence Synthetic oligonucleotide
acccagaagcagaaggtgggagaactgaag

SEQ ID NO: 86 - DNA, 41 bp, Artificial Sequence
  Description of Artificial Sequence Synthetic oligonucleotide
aaggacgagcaaatgtacctgcaccagaatcagaaggtggg

SEQ ID NO: 87 - DNA, 50 bp, Artificial Sequence
  Description of Artificial Sequence Synthetic oligonucleotide
ttcagttctcccaccttctgattctggtggtctactatccacgatttaac

SEQ ID NO: 88 - DNA, 30 bp, Artificial Sequence
  Description of Artificial Sequence Synthetic oligonucleotide
gtggttggagcatgtggtgttgggaaaagc

SEQ ID NO: 89 - DNA, 30 bp, Artificial Sequence
  Description of Artificial Sequence Synthetic oligonucleotide
gtggttggagcaggtggtgttgggaaaagc

SEQ ID NO: 90 - DNA, 42 bp, Artificial Sequence
  Description of Artificial Sequence Synthetic oligonucleotide
aaggacgagcaaatgtacctgcaggttggagcatgtggtgtt

SEQ ID NO: 91 - DNA, 50 bp, Artificial Sequence
  Description of Artificial Sequence Synthetic oligonucleotide
cttttcccaacaccacatgctccaacctggtctactatccacgatttaac

SEQ ID NO: 92 - DNA, 30 bp, Artificial Sequence
  Description of Artificial Sequence Synthetic oligonucleotide
gtggttggagcagatggtgttgggaaaagc

SEQ ID NO: 93 - DNA, 42 bp, Artificial Sequence
  Description of Artificial Sequence Synthetic oligonucleotide
aaggacgagcaaatgtacctgcaggttggagcagatggtgtt

SEQ ID NO: 94 - DNA, 50 bp, Artificial Sequence
  Description of Artificial Sequence Synthetic oligonucleotide
cttttcccaacaccatctgctccaacctggtctactatccacgatttaac

SEQ ID NO: 95 - DNA, 30 bp, Artificial Sequence
  Description of Artificial Sequence Synthetic oligonucleotide
tacaaactggtggtggttggagcaagtggt

SEQ ID NO: 96 - DNA, 30 bp, Artificial Sequence
  Description of Artificial Sequence Synthetic oligonucleotide
tacaaactggtggtggttggagcaggtggt

SEQ ID NO: 97 - DNA, 42 bp, Artificial Sequence
  Description of Artificial Sequence Synthetic oligonucleotide
aaggacgagcaaatgtacctgctcaaactggtggtggttgga

SEQ ID NO: 98 - DNA, 49 bp, Artificial Sequence
  Description of Artificial Sequence Synthetic oligonucleotide
cacttgctccaaccaccaccagtttgaggtctactatccacgatttaac

SEQ ID NO: 99 - DNA, 30 bp, Artificial Sequence
  Description of Artificial Sequence Synthetic oligonucleotide
gtggttggagcaggtgatgttgggaaaagc

SEQ ID NO: 100 - DNA, 42 bp, Artificial Sequence
  Description of Artificial Sequence Synthetic oligonucleotide
aaggacgagcaaatgtacctgcaggttggagcaggtgatgtt

SEQ ID NO: 101 - DNA, 50 bp, Artificial Sequence
  Description of Artificial Sequence Synthetic oligonucleotide
cttttcccaacatcacctgctccaacctggtctactatccacgatttaac

SEQ ID NO: 102 - DNA, 30 bp, Artificial Sequence
  Description of Artificial Sequence Synthetic oligonucleotide
atactggatacagctggacatgaagagtac

SEQ ID NO: 103 - DNA, 30 bp, Artificial Sequence
  Description of Artificial Sequence Synthetic oligonucleotide
atactggatacagctggacaagaagagtac

SEQ ID NO: 104 - DNA, 40 bp, Artificial Sequence
  Description of Artificial Sequence Synthetic oligonucleotide
aaggacgagcaaatgtacctgcaactggatacagctggac

SEQ ID NO: 105 - DNA, 49 bp, Artificial Sequence
  Description of Artificial Sequence Synthetic oligonucleotide
actcttcatgtccagctgtatccagttggtctactatccacgatttaac

SEQ ID NO: 106 - DNA, 30 bp, Artificial Sequence
  Description of Artificial Sequence Synthetic oligonucleotide
atactggatacagctggaaaagaagagtac

SEQ ID NO: 107 - DNA, 40 bp, Artificial Sequence
  Description of Artificial Sequence Synthetic oligonucleotide
aaggacgagcaaatgtacctgcaactggatacagctggaa

SEQ ID NO: 108 - DNA, 49 bp, Artificial Sequence
  Description of Artificial Sequence Synthetic oligonucleotide
actcttcttttccagctgtatccagttggtctactatccacgatttaac

SEQ ID NO: 109 - DNA, 30 bp, Artificial Sequence
  Description of Artificial Sequence Synthetic oligonucleotide
ggacatactggatacagctggactagaaga

SEQ ID NO: 110 - DNA, 30 bp, Artificial Sequence
  Description of Artificial Sequence Synthetic oligonucleotide
ggacatactggatacagctggacaagaaga

SEQ ID NO: 111 - DNA, 40 bp, Artificial Sequence
  Description of Artificial Sequence Synthetic oligonucleotide
aaggacgagcaaatgtacctgcaacatactggatacagct

SEQ ID NO: 112 - DNA, 49 bp, Artificial Sequence
  Description of Artificial Sequence Synthetic oligonucleotide
ttctagtccagctgtatccagtatgttggtctactatccacgatttaac

SEQ ID NO: 113 - DNA, 30 bp, Artificial Sequence
  Description of Artificial Sequence Synthetic oligonucleotide
atactggatacagctggacgagaagagtac

SEQ ID NO: 114 - DNA, 41 bp, Artificial Sequence
  Description of Artificial Sequence Synthetic oligonucleotide
aaggacgagcaaatgtacctgcaactggatacagctggacg

SEQ ID NO: 115 - DNA, 50 bp, Artificial Sequence
  Description of Artificial Sequence Synthetic oligonucleotide
tactcttctcgtccagctgtatccagttggtctactatccacgatttaac

SEQ ID NO: 116 - DNA, 30 bp, Artificial Sequence
  Description of Artificial Sequence Synthetic oligonucleotide
ctctctaaaatcactgagcaggagaaagat

SEQ ID NO: 117 - DNA, 30 bp, Artificial Sequence
  Description of Artificial Sequence Synthetic oligonucleotide
ctctctgaaatcactgagcaggagaaagat

SEQ ID NO: 118 - DNA, 41 bp, Artificial Sequence
  Description of Artificial Sequence Synthetic oligonucleotide
aaggacgagcaaatgtacctgcactctaaaatcactgagca

SEQ ID NO: 119 - DNA, 50 bp, Artificial Sequence
  Description of Artificial Sequence Synthetic oligonucleotide
tctttctcctgctcagtgattttagagtggtctactatccacgatttaac

SEQ ID NO: 120 - DNA, 30 bp, Artificial Sequence
  Description of Artificial Sequence Synthetic oligonucleotide
agatcctctctctgaaatcactaagcagga

SEQ ID NO: 121 - DNA, 30 bp, Artificial Sequence
  Description of Artificial Sequence Synthetic oligonucleotide
agatcctctctctgaaatcactgagcagga

SEQ ID NO: 122 - DNA, 42 bp, Artificial Sequence
  Description of Artificial Sequence Synthetic oligonucleotide
aaggacgagcaaatgtacctgcaatcctctctctgaaatcac

SEQ ID NO: 123 - DNA, 50 bp, Artificial Sequence
  Description of Artificial Sequence Synthetic oligonucleotide
cctgcttagtgatttcagagagaggattggtctactatccacgatttaac

SEQ ID NO: 124 - DNA, 30 bp, Artificial Sequence
  Description of Artificial Sequence Synthetic oligonucleotide
tgatgcacttcatggtggctggacaacaaa

SEQ ID NO: 125 - DNA, 30 bp, Artificial Sequence
  Description of Artificial Sequence Synthetic oligonucleotide
tgatgcacatcatggtggctggacaacaaa

SEQ ID NO: 126 - DNA, 41 bp, Artificial Sequence
  Description of Artificial Sequence Synthetic oligonucleotide
aaggacgagcaaatgtacctgcaatgcacttcatggtggct

SEQ ID NO: 127 - DNA, 49 bp, Artificial Sequence
  Description of Artificial Sequence Synthetic oligonucleotide
tgttgtccagccaccatgaagtgcattggtctactatccacgatttaac

SEQ ID NO: 128 - DNA, 30 bp, Artificial Sequence
  Description of Artificial Sequence Synthetic oligonucleotide
tgatgcacgtcatggtggctggacaacaaa

SEQ ID NO: 129 - DNA, 41 bp, Artificial Sequence
  Description of Artificial Sequence Synthetic oligonucleotide
aaggacgagcaaatgtacctgcaatgcacgtcatggtggct

SEQ ID NO: 130 - DNA, 49 bp, Artificial Sequence
  Description of Artificial Sequence Synthetic oligonucleotide
tgttgtccagccaccatgacgtgcattggtctactatccacgatttaac

SEQ ID NO: 131 - DNA, 30 bp, Artificial Sequence
  Description of Artificial Sequence Synthetic oligonucleotide
atcgactccaccgaggtcatctactagccg

SEQ ID NO: 132 - DNA, 30 bp, Artificial Sequence
  Description of Artificial Sequence Synthetic oligonucleotide
atcgactccaccgaggtcatctaccagccg

SEQ ID NO: 133 - DNA, 41 bp, Artificial Sequence
  Description of Artificial Sequence Synthetic oligonucleotide
aaggacgagcaaatgtacctgcacgactccaccgaggtcat

SEQ ID NO: 134 - DNA, 49 bp, Artificial Sequence
  Description of Artificial Sequence Synthetic oligonucleotide
gctggtagatgacctcggtggagtcgtggtctactatccacgatttaac

SEQ ID NO: 135 - DNA, 30 bp, Artificial Sequence
  Description of Artificial Sequence Synthetic oligonucleotide
atcccgggcgactgtggccccctgctctct

SEQ ID NO: 136 - DNA, 30 bp, Artificial Sequence
  Description of Artificial Sequence Synthetic oligonucleotide
atcccgggcgactgtggccccccgctctct

SEQ ID NO: 137 - DNA, 42 bp, Artificial Sequence
  Description of Artificial Sequence Synthetic oligonucleotide
aaggacgagcaaatgtacctgcacccgggcgactgtggcccc

SEQ ID NO: 138 - DNA, 49 bp, Artificial Sequence
  Description of Artificial Sequence Synthetic oligonucleotide
agagcagggggccacagtcgcccgggtggtctactatccacgatttaac

SEQ ID NO: 139 - DNA, 30 bp, Artificial Sequence
  Description of Artificial Sequence Synthetic oligonucleotide
aggacctcttggacatcgaggatgacatca

SEQ ID NO: 140 - DNA, 30 bp, Artificial Sequence
  Description of Artificial Sequence Synthetic oligonucleotide
aggacctcttcgacatcgaggatgacatca

SEQ ID NO: 141 - DNA, 41 bp, Artificial Sequence
  Description of Artificial Sequence Synthetic oligonucleotide
aaggacgagcaaatgtacctgcagacctcttggacatcgag

SEQ ID NO: 142 - DNA, 49 bp, Artificial Sequence
  Description of Artificial Sequence Synthetic oligonucleotide
atgtcatcctcgatgtccaagaggtctggtctactatccacgatttaac

SEQ ID NO: 143 - DNA, 30 bp, Artificial Sequence
  Description of Artificial Sequence Synthetic oligonucleotide
gttgtgaggcgctgcccccaccatgagcgc

SEQ ID NO: 144 - DNA, 30 bp, Artificial Sequence
  Description of Artificial Sequence Synthetic oligonucleotide
gttgtgaggcactgcccccaccatgagcgc

SEQ ID NO: 145 - DNA, 42 bp, Artificial Sequence
  Description of Artificial Sequence Synthetic oligonucleotide
aaggacgagcaaatgtacctgcatgtgaggcactgcccccac

SEQ ID NO: 146 - DNA, 49 bp, Artificial Sequence
  Description of Artificial Sequence Synthetic oligonucleotide
gctcatggtgggggcagtgcctcacatggtctactatccacgatttaac

SEQ ID NO: 147 - DNA, 30 bp, Artificial Sequence
  Description of Artificial Sequence Synthetic oligonucleotide
actttttgacatagtgtggtggtgccctat

SEQ ID NO: 148 - DNA, 30 bp, Artificial Sequence
  Description of Artificial Sequence Synthetic oligonucleotide
acttttcgacatagtgtggtggtgccctat

SEQ ID NO: 149 - DNA, 42 bp, Artificial Sequence
  Description of Artificial Sequence Synthetic oligonucleotide
aaggacgagcaaatgtacctgcatttttgacatagtgtggtg

SEQ ID NO: 150 - DNA, 49 bp, Artificial Sequence
  Description of Artificial Sequence Synthetic oligonucleotide
agggcaccaccacactatgtcaaaaatggtctactatccacgatttaac

SEQ ID NO: 151 - DNA, 30 bp, Artificial Sequence
  Description of Artificial Sequence Synthetic oligonucleotide
cgacatagtgtggtggtgccctgtgagccg

SEQ ID NO: 152 - DNA, 30 bp, Artificial Sequence
  Description of Artificial Sequence Synthetic oligonucleotide
cgacatagtgtggtggtgccctatgagccg

SEQ ID NO: 153 - DNA, 42 bp, Artificial Sequence
  Description of Artificial Sequence Synthetic oligonucleotide
aaggacgagcaaatgtacctgcaacatagtgtggtggtgccc

SEQ ID NO: 154 - DNA, 49 bp, Artificial Sequence
  Description of Artificial Sequence Synthetic oligonucleotide
gctcacagggcaccaccacactatgttggtctactatccacgatttaac

SEQ ID NO: 155 - DNA, 30 bp, Artificial Sequence
  Description of Artificial Sequence Synthetic oligonucleotide
ttcctgcatgggcggcatgaaccagaggcc

SEQ ID NO: 156 - DNA, 30 bp, Artificial Sequence
  Description of Artificial Sequence Synthetic oligonucleotide
ttcctgcatgggcggcatgaaccggaggcc

SEQ ID NO: 157 - DNA, 41 bp, Artificial Sequence
  Description of Artificial Sequence Synthetic oligonucleotide
aaggacgagcaaatgtacctgcacctgcatgggcggcatga

SEQ ID NO: 158 - DNA, 49 bp, Artificial Sequence
  Description of Artificial Sequence Synthetic oligonucleotide
cctctggttcatgccgcccatgcaggtggtctactatccacgatttaac

SEQ ID NO: 159 - DNA, 30 bp, Artificial Sequence
  Description of Artificial Sequence Synthetic oligonucleotide
atgaactggaggcccatcctcaccatcatc

SEQ ID NO: 160 - DNA, 30 bp, Artificial Sequence
  Description of Artificial Sequence Synthetic oligonucleotide
atgaaccggaggcccatcctcaccatcatc

SEQ ID NO: 161 - DNA, 41 bp, Artificial Sequence
  Description of Artificial Sequence Synthetic oligonucleotide
aaggacgagcaaatgtacctgcagaactggaggcccatcct

SEQ ID NO: 162 - DNA, 49 bp, Artificial Sequence
  Description of Artificial Sequence Synthetic oligonucleotide
tgatggtgaggatgggcctccagttctggtctactatccacgatttaac

SEQ ID NO: 163 - DNA, 30 bp, Artificial Sequence
  Description of Artificial Sequence Synthetic oligonucleotide
aggtgcatgtttgtgcctgtcctgggagag

SEQ ID NO: 164 - DNA, 30 bp, Artificial Sequence
  Description of Artificial Sequence Synthetic oligonucleotide
aggtgcgtgtttgtgcctgtcctgggagag

SEQ ID NO: 165 - DNA, 41 bp, Artificial Sequence
  Description of Artificial Sequence Synthetic oligonucleotide
aaggacgagcaaatgtacctgcagtgcatgtttgtgcctgt

SEQ ID NO: 166 - DNA, 49 bp, Artificial Sequence
  Description of Artificial Sequence Synthetic oligonucleotide
ctcccaggacaggcacaaacatgcactggtctactatccacgatttaac

SEQ ID NO: 167 - DNA, 30 bp, Artificial Sequence
  Description of Artificial Sequence Synthetic oligonucleotide
gggagagactggcgcacagaggaagagaat

SEQ ID NO: 168 - DNA, 30 bp, Artificial Sequence
  Description of Artificial Sequence Synthetic oligonucleotide
gggagagaccggcgcacagaggaagagaat

SEQ ID NO: 169 - DNA, 41 bp, Artificial Sequence
  Description of Artificial Sequence Synthetic oligonucleotide
aaggacgagcaaatgtacctgcagagagactggcgcacaga

SEQ ID NO: 170 - DNA, 49 bp, Artificial Sequence
  Description of Artificial Sequence Synthetic oligonucleotide
tctcttcctctgtgcgccagtctctctggtctactatccacgatttaac

SEQ ID NO: 171 - DNA, 30 bp, Artificial Sequence
  Description of Artificial Sequence Synthetic oligonucleotide
gtgcgaacacaggtacatttgctcgtcctt

SEQ ID NO: 172 - DNA, 39 bp, Artificial Sequence
  Description of Artificial Sequence Synthetic oligonucleotide
aaggacgagcaaatgtacctgggaacagctttgaggtgt

SEQ ID NO: 173 - DNA, 47 bp, Artificial Sequence
  Description of Artificial Sequence Synthetic oligonucleotide
acaaacacacacctcaaagctgttccgtctactatccacgatttaac

SEQ ID NO: 174 - DNA, 30 bp, Artificial Sequence
  Description of Artificial Sequence Synthetic oligonucleotide
gttaaatcgtggatagtagactgttcgcac

SEQ ID NO: 175 - DNA, 26 bp, Artificial Sequence
  Description of Artificial Sequence Synthetic oligonucleotide
gtgcgcaggtacatttgctcgtcctt

SEQ ID NO: 176 - DNA, 43 bp, Artificial Sequence
  Description of Artificial Sequence Synthetic oligonucleotide
aaggacgagcaaatgtacctgaacaggaacagctttgaggtgt

SEQ ID NO: 177 - DNA, 51 bp, Artificial Sequence
  Description of Artificial Sequence Synthetic oligonucleotide
acaaacacacacctcaaagctgttcctgttgtctactatccacgatttaac

SEQ ID NO: 178 - DNA, 26 bp, Artificial Sequence
  Description of Artificial Sequence Synthetic oligonucleotide
gttaaatcgtggatagtagaccgcac

SEQ ID NO: 179 - DNA, 45 bp, Artificial Sequence
  Description of Artificial Sequence Synthetic oligonucleotide
aaggacgagcaaatgtacctgcgaacaggaacagctttgaggtgt

SEQ ID NO: 180 - DNA, 53 bp, Artificial Sequence
  Description of Artificial Sequence Synthetic oligonucleotide
acaaacacacacctcaaagctgttcctgttcggtctactatccacgatttaac

(19) United States
(12) Patent Application Publication
(10) Pub. No.: US 2016/0326600 A1
(43) Pub. Date: Nov. 10, 2016

(54) ECONOMICAL MOLECULES FOR SPECIFIC BINDING AND DETECTION OF NUCLEIC ACIDS USING UNIVERSAL FUNCTIONALIZED STRANDS

(71) Applicant: WILLIAM MARSH RICE UNIVERSITY, HOUSTON, TX (US)

(72) Inventors: DAVID YU ZHANG, HOUSTON, TX (US); JUEXIAO WANG, HOUSTON, TX (US)

(21) Appl. No.: 15/216,214

(22) Filed: Jul. 21, 2016

Related U.S. Application Data
(63) Continuation of application No. PCT/US15/13450, filed on Jan. 29, 2015.
(60) Provisional application No. 61/932,898, filed on Jan. 29, 2014.

(51) Int. Cl. C12Q 1/68

(52) U.S. Cl. CPC C12Q 1/6886; C12Q 2600/156

(57) ABSTRACT

Nucleic acid probes are described comprising a universal component and a target dependent component. The universal component provides an economical advantage in that the universal component can retain any desired functional moiety, such as a fluorophore or other label which can be used with any target dependent component. Thus, the cost of designing target specific functionalized probes is significantly reduced with this probe system by limiting de novo synthesis to only the target dependent component of the probe for each desired target nucleic acid. Furthermore, the probe design of the present disclosure provides high target specificity demonstrating selective binding of the target in a 1% target load sample.

### CROSS REFERENCE TO RELATED APPLICATIONS

The present application is a continuation application of International Application No. PCT/US15/13450, filed Jan. 29, 2015, which claims priority to U.S. Provisional Application No. 61/932,898 filed Jan. 29, 2014, which is incorporated herein by reference in its entirety.

### STATEMENT OF GOVERNMENT INTEREST

This invention was made with government support under Award No. EB015331 awarded by NIH National Institute of Biomedical Imaging and Bioengineering (NIBIB). The government has certain rights in the invention.

### SEQUENCE LISTING

The instant application contains a Sequence Listing which has been submitted electronically in ASCII format and is hereby incorporated by reference in its entirety. Said ASCII copy, created on Jan. 23, 2015, is named 14-21001-WO(260947.00244)_SL.txt and is 42,800 bytes in size.

### BACKGROUND

The detection of specific DNA and RNA sequences has many uses in clinical diagnostic, scientific research, and agricultural markets. For example, the detection of cancer driver mutations, bacteria/virus subtypes, and single nucleotide polymorphism (SNP) is important for both health and research purposes. The detection methods could either use standard PCR assays to detect the sequence of interest from a well-mixed solution, or they could use in situ hybridization (ISH) approaches that provide the localization patterns of DNA and RNA. In the agricultural industry, nucleic acid detection could be used for genotyping viruses and bacteria that may potentially lead to foodborne diseases (e.g. Salmonella), and for identifying genetically modified organisms (GMO).

One major limitation of many previous DNA and RNA detection technologies is the requirement of expensive equipment and/or reagents. The development of inexpensive reagents that could be used in conjunction with inexpensive equipment would improve the throughput of scientific progress, facilitate inexpensive clinical diagnostics, and encourage more consistent agricultural screening.

A large driver of reagent cost is the need to use different functionalized oligonucleotides for quantitation and/or visualization of different target sequences. Large pools of functionalized oligonucleotides are expensive to synthesize because of low synthesis yield and purification steps, but are frequently used in nucleic acid biotechnologies. Therefore, a universal functionalized sequence that can be used for the assaying of a variety of nucleic acid sequences would significantly reduce the reagent cost.

### SUMMARY

The probe system of the present disclosure comprises a universal component and a target dependent component. The universal component comprises at least a first universal oligonucleotide/strand and in some embodiments, a second universal oligonucleotide/strand. The first and second universal strands each comprise one region and in some embodiments, two regions. The first and second universal strands comprise sequence that is not target specific and therefore can be used with any target dependent component. The target dependent component comprises a protector strand and a target-specific/complement strand. The protector strand comprises at least one region and in some embodiments, two or more regions. One or more regions of the protector strand is at least partially complementary (and in some instances fully complementary) to at least a portion of the second universal strand and can form a first double-stranded region. The target-specific strand (i.e. complement strand) comprises at least two regions and in some instances three or more regions. At least one of the regions of the target-specific strand is fully or partially complementary to the at least one region of the first universal strand and may give rise to a second double-stranded region. In some instances, the protector strand has a region that is at least partially complementary (and in some instances fully complementary) to all or a portion of the target-specific strand and may give rise to a third double-stranded region. The target-specific strand contains another region that is not hybridized to any other strand of the probe, but is complementary to a portion of the target sequence. To be clear, the region of the target-specific strand that is complementary to a region of the protector strand is also complementary to the target sequence. The first universal strand may comprise a further region that is fully or partially complementary to the second universal strand which may give rise to a fourth double-stranded region. In some embodiments, the first universal strand comprises a label conjugated thereto. In other embodiments, the second universal strand comprises a moiety that prevents detection of the label prior to hybridization of the probe to the target sequence.

Upon hybridization of the probe to the target nucleic acid, the protector strand and any universal strand hybridized thereto dissociates from the target-specific strand leaving the target-specific strand, along with any universal strand hybridized thereto, hybridized to the target nucleic acid. Thus, the probes of the present disclosure permit the use of functionalized universal components with a variety of target dependent components thereby eliminating the expense of synthesizing a different functionalized probe for each desired target sequence.

### BRIEF DESCRIPTION OF THE DRAWINGS

FIG. 1 provides four exemplary probes (referred to herein as the X-probe) of the present disclosure. Referring now to (a), one embodiment of the X-probe comprises four (4) nucleic acid strands: first universal strand A; second universal strand B; protector strand P; and complement strand C (also referred to herein as the “target-specific strand”). Each strand is subdivided into regions comprising contiguous nucleotide bases, denoted by reference numbers 1-6 and 10-13, wherein region 1 of second universal strand B is also referred to as “first region,” region 2 of the protector strand P is also referred to as “second region” and so on for the other regions. The sequences of opposing regions (e.g. region 7 of strand A and region 6 of strand C) are complementary or at least partially complementary. Referring now to (b), the X-probe components may further comprise multiple optional regions (region 10, 11, 12, and 13). Panels (c) and (d) show X-probe designs comprising only one universal strand. The arrow at one end of the strand represents that the end is the 3′ of the oligonucleotide (e.g. the sixth region exists to the 3′ of the fifth region). For any of these embodiments, a functionalized moiety (not shown) can be conjugated to the 5′ end, to the 3′ end, or to an internal nucleotide of the first universal strand and the second universal strand. Prior to reacting with target nucleic acids strand, the various probe components are hybridized together.

FIG. 2 provides an exemplary reaction of one embodiment of the present X-probe with target nucleic acid T. Upon the X-probe reacting with the target T, the BP complex (comprising strands B and P) of the probe dissociates from the AC complex which is hybridized to target T.

FIG. 3 provides an exemplary reaction of one embodiment of the X-probe wherein the X-probe provides conditional fluorescence upon hybridization to target. In this embodiment, the second universal strand B is functionalized with a quencher, and the first universal strand A is functionalized with a fluorophore. The X-probe is natively dark because the fluorophore is in close proximity to the quencher. Upon hybridization the probe to target T, the quencher-containing complex AP is displaced and diffuses away, and the fluorophore of the BC complex bound to target T is exposed thereby increasing the fluorescence. Thus, fluorescence signal indicates the existence and implies the concentration of the target T in solution.

FIG. 4 provides an exemplary process for preparing one embodiment of a probe of the present disclosure. On the left panel, four components at the indicated concentrations (B at 5×, P at 3×, A at 1×, and C at 1.5×) are mixed and thermally annealed before operation. The concentration of each strand relative to the limiting reagent (lx A) is shown. On the right panel, after annealing, the probe mixture comprises several different multi-stranded complexes. The concentrations of the four strands are determined in a way such that the probe mixture has minimum native fluorescence (resulting from fluorophore F on strand) and minimum number of multi-stranded species.

FIG. 5 provides an exemplary probe of the present disclosure and a target nucleic acid comprising SEQ ID. NO. 1 used in Example 1. The probe was designed such that universal strand A (region 7) has SEQ ID. NO. 2 with a functional ROX group conjugated to the 3′ end, universal strand B (region 1) has SEQ ID. NO. 3 with a quencher RQ conjugated to the 5′ end, protector strand P (regions 2 and 3) has SEQ ID. NO. 4, and complementary strand (regions 4, 5, and 6) has SEQ ID. NO. 5. The inset shows the experimentally observed time-based fluorescence response upon the addition of the target nucleic acid to a sample containing the probe as described in Example 1.

FIG. 6 provides an example of the standard free energy (ΔG°na) calculations used for design of the probes of the present disclosure. Energy terms with subscripted numbers represent the standard free energy of hybridization between referenced regions (e.g. ΔG°₉₋₄ represents the standard free energy of hybridization between region 9 of the target and region 4 of the complement strand C of the probe).

FIG. 7 provides standard free energy values for four examples of the probes of the present disclosure with each probe having optional regions of varying length (regions 10, 11, 12, and 13). All energies were calculated at 37° C., in 0.75 M Na+. Between (a)-(d), regions 1-7of the probe are unchanged where region 1 has SEQ ID NO. 6, region 2 has SEQ ID NO. 7, region 3 has SEQ ID NO. 8, region 4 has a nucleotide sequence of 5′ . . . acaaacac . . . , region 5 has SEQ ID NO. 9, region 6 has SEQ ID NO. 10, and region 7 has SEQ ID NO. 11. In (a), probe does not include regions 12 and 13, and region 10 has a sequence of 5′gtgcgaaca . . . 3′ and region 11 has a sequence of 5′ . . . tgttcgcac 3′. In (b), the probe includes regions 12 and 13 comprising two nucleotides each, 5′ . . . ca . . . 3′ and 5′ . . . tg . . . 3′, respectively. Regions 10 and 11 in (b) are shorter than that in (a) and region 10 in (b) has a sequence of 5′gtgcgaa . . . 3′ and region 11 has a sequence of 5′ . . . ttcgcac 3′. In (c), regions 10 and 11 are shorter than the probe in (a) and (b), where in (c) region 10 has a sequence of 5′gtgcg . . . 3′ and region 11 has a sequence of 5′ . . . cgcac 3′. Furthermore, in (c), regions 12 and 13 are each two nucleotides longer than in (b) and having sequences of 5′ . . . aaca . . . 3′ and 5′ . . . tgtt . . . 3′, respectively. Referring now to (d), regions 10 and 11 are omitted from universal strand B and A, respectively. Regions 12 and 13 include 2 additional nucleotides from that in (c) such that the sequences are 5′ . . . cgaaca . . . 3′ and 5′ . . . tgttcg . . . 3′, respectively. Target T has SEQ ID NO. 12. In (a), regions 10 and 1 encompass SEQ ID NO: 171, regions 2 and 3, encompass SEQ ID NO: 172, regions 4, 5, and 6, encompass SEQ ID NO: 173, and regions 7 and 11 encompass SEQ ID NO: 174. In (b), regions 10 and 1 encompass SEQ ID NO: 14, regions 2, 12, and 3 encompass SEQ ID NO: 15, regions 4, 5, 13, and 6 encompass SEQ ID NO: 16, and regions 7 and 11 encompass SEQ ID NO: 13. In (c), regions 10 and 1 encompass SEQ ID NO: 175, regions 2, 12, and 3 encompass SEQ ID NO: 176, regions 4, 5, 13, and 6 encompass SEQ ID NO: 177, and regions 7 and 11 encompass SEQ ID NO: 178. In (d), regions 2, 12, and 3 encompass SEQ ID NO: 179 and regions 4, 5, 13, and 6 encompass SEQ ID NO: 180.

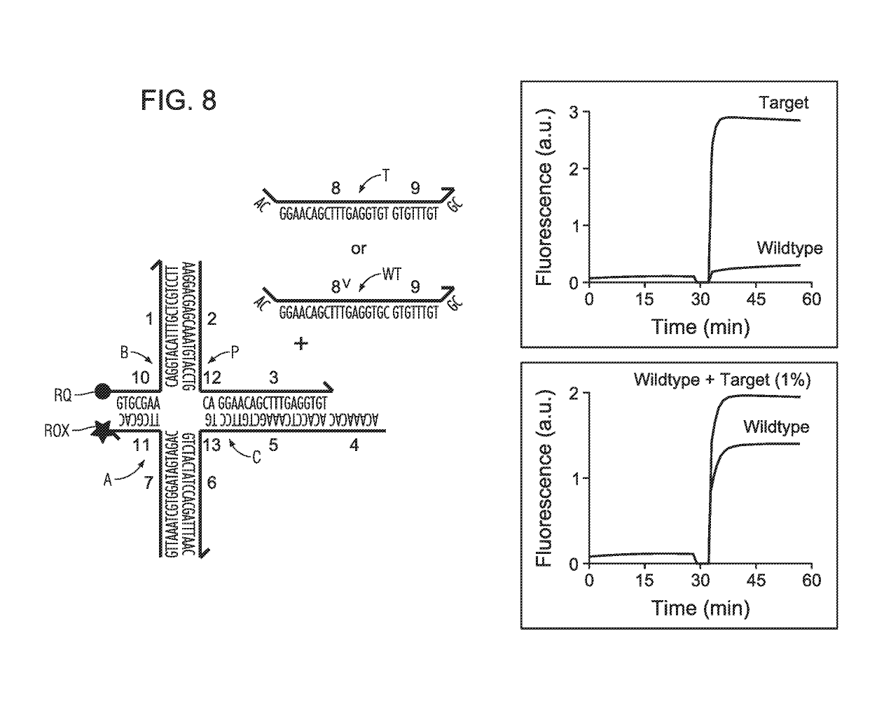

FIG. 8 demonstrates the specificity of one exemplary probe of the present disclosure targeting the TP53 R273C (c.817C>T) mutation (in region 8 of the target and wild-type—where region 8 is denoted 8°)) in samples containing equal concentrations of nucleic acid containing the mutation and wild type (upper right inset) and in samples containing only 1% of nucleic acid containing the mutation (lower right inset). The probe comprises universal strand A (regions 7 and 11) of SEQ ID NO. 13 having a carboxy-X-rhodamine fluorophore (ROX) conjugated to the 3′ end, universal strand B (regions 1 and 10) of SEQ ID NO. 14 having an Iowa Black Red Quencher (RQ) conjugated to the 5′ end, protector strand P (regions 2, 12, and 3) of SEQ ID NO. 15, and complement strand C (regions 4, 5, 13, and 6) of SEQ ID NO. 16. It should be noted that regions 11 and 10 of universal strands A and B comprise 7 nucleotides (the terminal seven 3′ and 5′ nucleotides, respectively). Target T harboring the above described mutation has SEQ ID NO. 17 whereas the wild-type WT has SEQ ID NO. 18.

FIG. 9 demonstrates the specificity of one exemplary probe of the present disclosure targeting the EGFR L858R (c.2573T>G) mutation (in region 8 of the target—indicated by super scripted “v”) in samples containing equal concentrations of nucleic acid containing the mutation and wild type (upper right inset) and in samples containing only 1% of nucleic acid containing the mutation (lower right inset). The probe comprises universal strand A (regions 7 and 11) of SEQ ID NO. 13 having a carboxy-X-rhodamine fluorophore (ROX) conjugated to the 3′ end, universal strand B (regions 1 and 10) of SEQ ID NO. 14 having an Iowa Black Red Quencher (RQ) conjugated to the 5′ end, protector strand P (regions 2, 12, and 3) of SEQ ID NO. 19, and complement strand C (regions 4, 5, 13, and 6) of SEQ ID NO. 20. It should be noted that regions 11 and 10 of universal strands A and B comprise 7 nucleotides (the terminal seven 3′ and 5′ nucleotides, respectively). Target T harboring the above described mutation has SEQ ID NO. 21 whereas the wild-type WT has SEQ ID NO. 22.

FIG. 10 demonstrates the specificity of one exemplary probe of the present disclosure targeting the KRAS Q61H (c.183A>C) mutation (in region 8 of the target—indicated by super scripted “v”) in samples containing equal concentrations of nucleic acid containing the mutation and wild type (upper right inset) and in samples containing only 1% of nucleic acid containing the mutation (lower right inset). The probe comprises universal strand A (regions 7 and 11) of SEQ ID NO. 13 having a carboxy-X-rhodamine fluorophore (ROX) conjugated to the 3′ end, universal strand B (regions 1 and 10) of SEQ ID NO. 14 having an Iowa Black Red Quencher (RQ) conjugated to the 5′ end, protector strand P (regions 2, 12, and 3) of SEQ ID NO. 23, and complement strand C (regions 4, 5, 13, and 6) of SEQ ID NO. 24. It should be noted that regions 11 and 10 of universal strands A and B comprise 7 nucleotides (the terminal seven 3′ and 5′ nucleotides, respectively). Target T harboring the above described mutation has SEQ ID NO. 25 whereas the wild-type WT has SEQ ID NO. 26.

FIG. 11 provides a summary of experimental results for 44 exemplary probes of the present disclosure (sequences for the probes, targets, and wild types described in Tables 1-10) targeting 44 different cancer-relevant single nucleotide variants (SNV) in samples containing 1% of the nucleic acid carrying the SNP (target) where the remaining nucleic acid in the sample being synthetic oligonucleotides carrying the wild type (WT) sequence.

FIG. 12 provides one embodiment of the present disclosure wherein a single firsts universal strand of the probe is provided in a sample with three different target recognition regions of the probe (P1/C1, P2/C2, P3/C3) such that the presence of multiple target nucleic acids (T1, T2, T3) can be detected in a single sample with fluorescence responses for each having the same spectral properties.

FIG. 13 provides another embodiment of the present disclosure wherein multiple probes each possessing different functional groups (Alexa488, Alexa532, Alexa647) and each directed to a different nucleic acid target are applied to a sample to detect and identify the presence of the different targets simultaneously.

FIG. 14 provides embodiments of the present probe comprising different variations which do not require moving or reorganizing the components of the probe. The variations depicted include: inverted 5′/3′ orientation (A); types and positions of functionalization (B); multi-loop configurations (C); and mismatches (D).

FIG. 15 provides embodiments of the present probe comprising further variations which may require additional auxiliary components or regions, reorganizing the original components of the X-probe, and/or changing the connections between regions. The variations depicted include: increased arms and connection morphology (A); morphology changes of the universal strands (B); and reduced designs (C).

### DESCRIPTION

The present disclosure generally relates to nucleic acid probes, and more specifically, to nucleic acid probes comprising a universal component and a target dependent component.

One advantage of the present probe system is that the universal oligonucleotide is decoupled from the target nucleic acid specificity such that the same universal oligonucleotide can be produced for probes having specificity for a variety of related or unrelated nucleic acid sequences. Furthermore, the sequences of the universal component (and the regions of the target dependent component that complement the universal component) are formed of arbitrary sequence to avoid the formation of unintended hybridization products with other nucleic acids in the sample. The term “arbitrary” is only intended to mean that the sequence is designed to not hybridize with any nucleic acid molecules in a sample beyond the nucleic acid strands/oligonucleotides used to form the probe.

The target dependent component is synthesized for the desired target without requiring specialized synthesis of the universal component, which in many instances may comprise a functional moiety such as a label, thereby significantly decreasing the expense associated with producing multiple functionalized probes for each desired target sequence. In other words, the probes of the present disclosure only require modification of the target dependent component sequences with respect to each desired target, and the universal component sequences can be kept constant regardless of the target dependent component used. The universal component can comprise conserved sequence that is capable of hybridization with conserved regions of the target dependent component. These conserved regions of the universal component and target dependent component are not specific to the target nucleic acid sequence and are generally arbitrary with respect to the genetic material of the organism being tested (i.e., should not be capable of hybridizing to any other nucleic acids present in the sample). Target specificity is therefore achieved by providing target specific regions in the target dependent component.

In another embodiment, the present disclosure provides a nucleic acid probe comprising a universal functionalized component and a target dependent component such that the probe can be used to detect a large number of target nucleic acids without requiring synthesis of multiple species of functionalized probe. Instead, the target dependent component of the present probe can be modified to provide specificity to the target without requiring modification to the functionalized component thereby providing a more economical approach to probe synthesis.

In one embodiment, the universal component comprises a first universal nucleic acid strand (i.e. first universal oligonucleotide) and a second universal nucleic acid strand (i.e. second universal oligonucleotide) wherein the second universal nucleic acid strand includes at least one region and the first universal strand includes at least one region. In some instances, the universal strand further comprises at least one functional moiety to permit detection of the probe. As discussed further herein, the universal component may comprise only a first universal nucleic acid strand.

In one embodiment, the target dependent component comprises a protector nucleic acid strand (i.e. protector oligonucleotide) and a complement nucleic acid strand (i.e. complement oligonucleotide) wherein the protector strand includes at least one region and the complement strand includes at least two regions. The complement strand, in this embodiment, provides the sequence that is specific for hybridization of the probe to the target nucleic acid sequence.

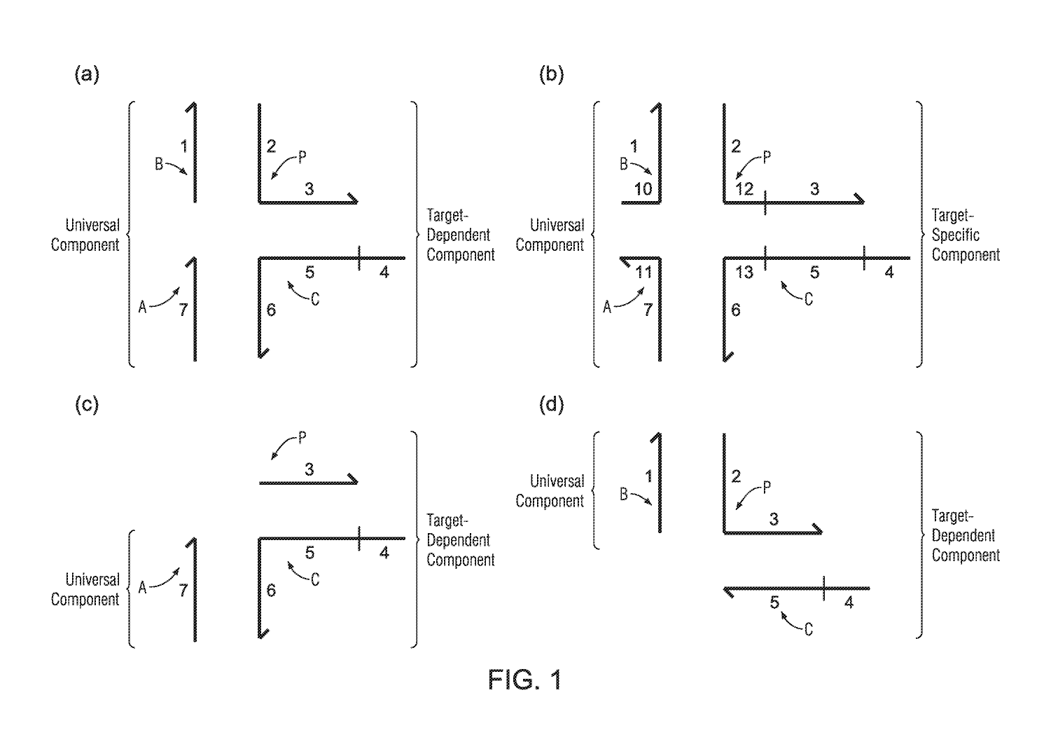

Referring now to FIG. 1, examples of a probe of the present disclosure is provided (also referred to herein as “X-probe”). In panels a and b, the X-probe comprises four oligonucleotides or strands including the first universal strand A, the second universal strand B, the complement strand C, and the protector strand P. Each oligonucleotide is divided into one or more regions with patterns of interactions and complementarities as described herein. Each region, represented by a number, denotes a portion of an oligonucleotide that functionally acts as a unit in hybridization or dissociation. For example, the second universal strand B comprises region 1, the first universal strand A comprises region 7, the complement strand C comprises regions 4, 5, and 6, and the protector strand P comprises regions 2 and 3. One region is said to be complementary to another region if the nucleotides of each region can simultaneously form several Watson-Crick base pairs with each other. To this end, the strands are designed such that region 1 is partially or fully complementary to region 2 thereby forming a first double-stranded region of the probe, region 6 is partially or fully complementary to region 7 thereby forming a second double-stranded region of the probe, and region 3 is partially or fully complementary to region 5 thereby forming at least a portion of a third double-stranded region of the probe. Additionally, the second universal strand B can comprise optional region 10, and the first universal strand A can comprise optional region 11, such that region 10 is partially or fully complementary to region 11 thereby forming an optional fourth double-stranded region of the probe. The protector strand P can comprise optional region 12, and the complement strand C can comprise optional region 13, such that region 12 is partially or fully complementary to region 13 which forms a portion of the third double-stranded region described above. These additional regions can confer important functional properties to the X-probe, such as reduced fluorescence background (in the case of a conditionally fluorescent X-probe) and/or high specificity.

Still referring to FIG. 1, in (c) and (d), the X-probe comprises only three oligonucleotides or strands, including either the first universal strand A or the second universal strand B, the complement strand C, and the protector strand P. The reduced designs also function in the spirit of the X-probes.

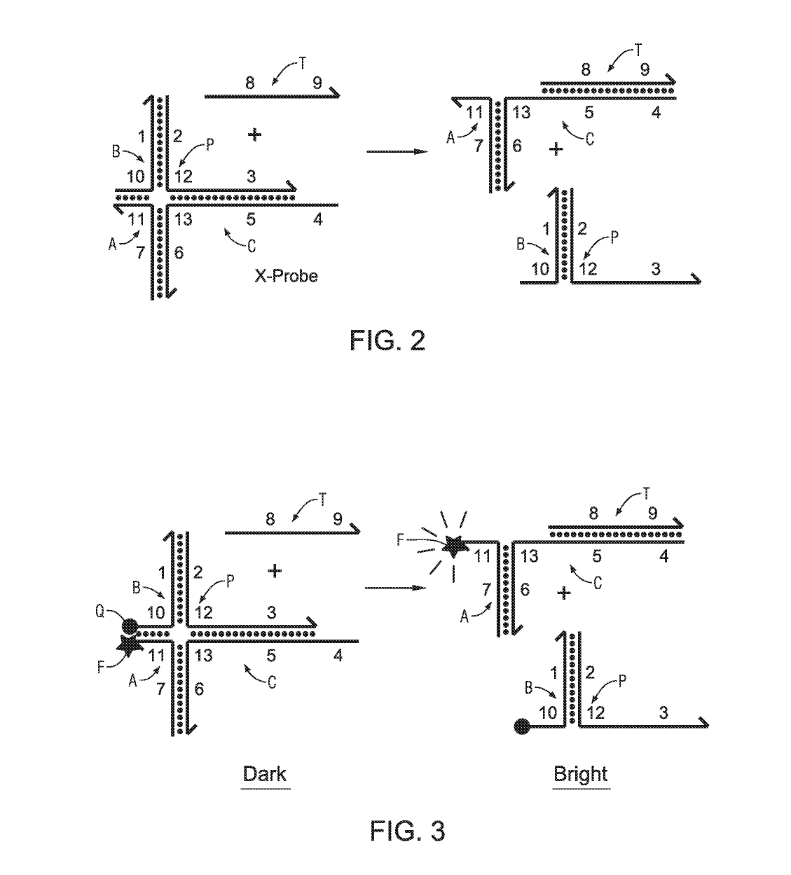

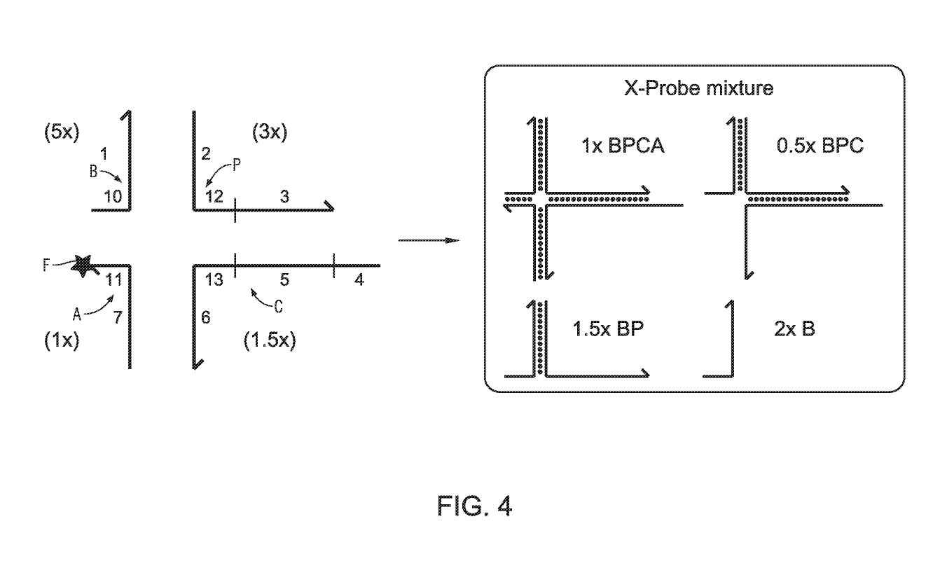

Referring now to FIG. 2, a probe such as that described above in (b) of FIG. 1 is shown along with a target nucleic acid comprising regions 8 and 9. Although not depicted as being embedded in a larger nucleic acid molecule, target T will in most instances simply comprise the target sequence of a larger nucleic acid molecule. As the probe is introduced into a sample containing target T and under the proper temperature and buffer conditions, regions 4 and 5 of the complement strand hybridize to regions 9 and 8, respectively, of the target T. Upon such hybridization, region 7 of universal strand A will remain hybridized to region 6 of complement strand C (“AC complex” or “complex AC”) whereas protector strand P and universal strand B (the “BP complex” or “complex BP”) will dissociate from the AC complex. As shown in FIG. 3, in the instance a fluorophore F is conjugated to the 3′ end of universal strand A (region 11) and a quencher Q is conjugated to the 5′ end of universal strand B (region 10), the dissociation of the BP complex from the AC complex upon binding to the target will remove the quencher Q and allow the fluorophore F to fluoresce as universal strand A remains hybridized to region 6 of complement strand C.

In one embodiment, the nucleotide sequence of the first region of the second universal strand is at least 60% complementary to the nucleotide sequence of the second region of the protector strand. In this or other embodiments, the nucleotide sequence of the third region of the protector strand is at least 60% complementary to the nucleotide sequence of the fifth region of the complement strand. In these or other embodiments, the nucleotide sequence of the sixth region of the component strand is at least 60% complementary to the nucleotide sequence of the seventh region of the first universal strand. In these or other embodiments, the nucleotide sequence of the first region of the second universal strand is less than 60% complementary to the nucleotide sequence of the seventh region of the first universal strand. In these or other embodiments, the nucleotide sequence of the second region of the protector strand is less than 60% complementary to the nucleotide sequence of the sixth region of the component strand.

In any of the above embodiments, the probe is designed to hybridize to a target nucleic acid sequence T comprising an eighth region and a ninth region as shown in FIG. 2. In these embodiments, the nucleotides of the eighth region of target T is at least 60% complementary to the nucleotides of the fifth region of the complement strand and the nucleotides of the ninth region of target T is at least 60% complementary to the nucleotides of the fourth region of the complement strand. The sequences of the first, second, sixth, and seventh regions are arbitrary and do not depend on the sequence of the target. Accordingly, multiple functionalized probes can be synthesized by modifying the third region of the protector strand and the fourth and fifth regions of the complement strand thereby providing an economically-efficient probe system.

In any of the above embodiments, upon the X-probe reacting with the target T, complex AC is colocalized with the target. The complex AC is also referred to herein as a target binding component.

In any of the above embodiments, upon the X-probe reacting with the target T, pre-hybridized complex BP that is displaced by the target T is also referred to herein as a protector component.

As used herein, the term “protector” when used to describe a nucleic acid strand, means that such nucleic acid strand is hybridized to the target-binding strand or a portion thereof and is displaced upon hybridization of the complement strand with the target nucleic acid or target nucleotide sequence.

As used herein, the term “region” when referring to the probe means subsequences within the designated strand and should not be understood to denote separate oligonucleotides.

In the above embodiments, the various strands of the present probe may each further comprise one or more additional regions. For example, the second universal strand may further comprise a tenth region and the first universal strand may further comprise an eleventh region wherein at least 60% of the nucleotides of the tenth region are complementary to the aligned nucleotides of the eleventh region. The sequences of the tenth and the eleventh regions, in this instance, are arbitrary and do not depend on the sequence of the target. In another example, the protector strand further comprises a twelfth region between the second region and the third region, and the complement strand further comprises a thirteenth region between the fifth region and the sixth region wherein at least 60% of the nucleotides of the twelfth region are complementary to the aligned nucleotides of the thirteenth region. The sequences of the twelfth and the thirteenth regions, in this instance, are arbitrary and do not depend on the sequence of the target.

In one embodiment, a probe of the present disclosure as described above further comprising a tenth and eleventh region, a first label on the first universal strand and a second label on the second universal strand, and a multi-loop formed at the junction of a different hybridized regions possesses a standard free energy of the hybridization reaction ΔG°_{r×n} to a target nucleic acid sequence that is calculated as:

ΔG°_{r×n}ΔG°₉₋₄−ΔG°₁₀₋₁₁−ΔG°_{ML}+(ΔG°₈₋₅−ΔG°₃₋₅)−ΔG°_{label}

wherein ΔG°₉₄ is the standard free energy of the hybridization between the ninth region and the fourth region, is the standard free energy of the hybridization between the tenth region and eleventh region, wherein ΔG°₈₋₅ is the standard free energy of the hybridization between the eighth region and the fifth region, wherein ΔG°₃₋₅ is the standard free energy of the hybridization between the third region and the fifth region, wherein ΔG°_{ML} is the standard free energy of the multi-loop formed at the junction of different hybridized regions, and ΔG°_{label} is the standard free energy difference between the thermodynamic contribution of the labels on the first universal strand when they are in close proximity of the second universal strand versus when they are delocalized. In another embodiment, the probe comprises an twelfth and thirteenth region instead of the tenth and eleventh region and does not include a multi-loop such that the probe possesses a standard free energy of the hybridization reaction ΔG°_{r×n} to a target nucleic acid sequence that is calculated as:

ΔG°_{r×n}=ΔG°₉₋₄−ΔG°₁₂₋₁₃+(ΔG°₈₋₅−ΔG°₃₋₅)−ΔG°_{label}

wherein ΔG°₁₂₋₁₃ is the standard free energy of hybridization between the twelfth region and the thirteenth region.

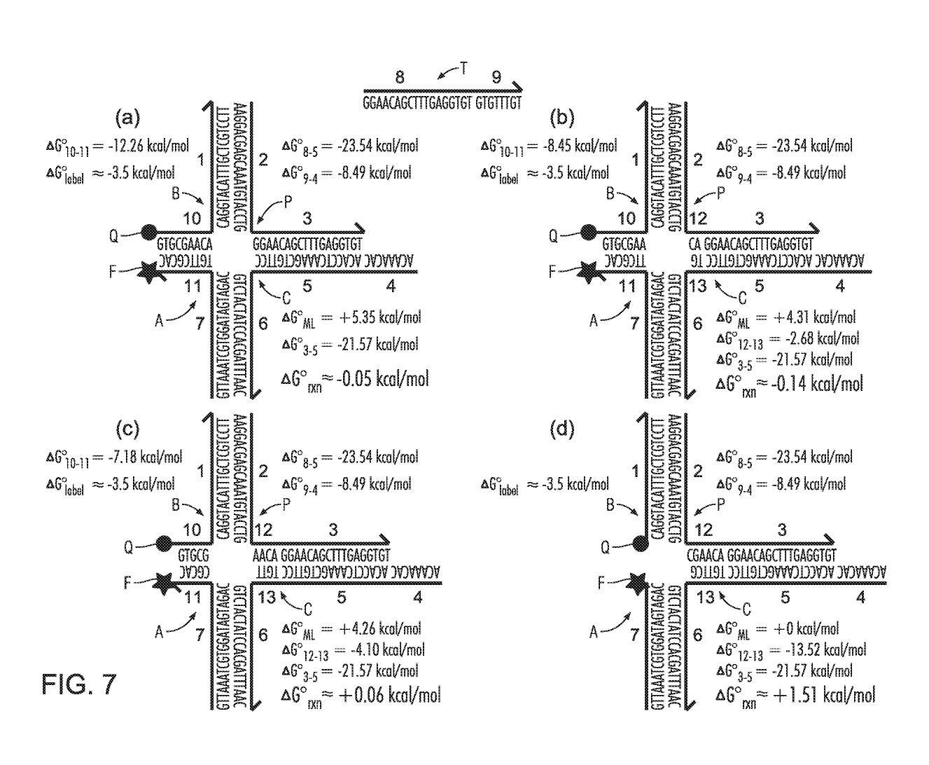

In yet another embodiment depicted in FIG. 6, a probe of the present disclosure comprising a tenth and eleventh region, a twelfth and thirteenth region, a first label on the first universal strand and a second label on the second universal strand, and a multi-loop (ML) formed at the junction of the different hybridized regions possesses a standard free energy of the hybridization reaction ΔG°_{r×n} to a target nucleic acid sequence that is calculated as:

ΔG°_{r×n}=ΔG°₉₋₄−ΔG°₁₀₋₁₁−ΔG°₁₂₋₁₃−ΔG°_{ML}+(ΔG°₈₋₅−ΔG°₃₋₅)−ΔG°_{label}

It should be understood that the calculation of the standard free energy of hybridization ΔG°_{r×n} can be modified from what is provided herein based on the absence or presence of optional regions and structural features, such as the tenth and eleventh regions and the multi-loop structure, which are only present in certain embodiments of the present probe. Thus, the equations provided herein may be modified to not include these terms. Similarly, various other structures or regions not described herein may be added to the probe such that the standard free energy calculations may need to include other terms to account for such changes. The description provided herein for calculating the standard free energy is sufficient to permit one skilled in the art to make such modifications to the calculations in view of any such changes in probe structure.

In any of the above embodiments, the sequences of the regions can be designed such that the interaction between the probe and the target possesses a standard free energy ΔG°_{r×n} within −4 kcal/mol to +4 kcal/mol, or a standard free energy that determined by ΔG°_{r×n} is within 5 kcal/mol of the standard free energy that determined by equation −Rτ ln([BP]/[BPCA]), wherein R is the ideal gas constant, τ is temperature in Kelvin, [BP] is the final concentration of complex BP in X-probe solution, and [BPCA] is the final concentration of the X-probe in solution. For embodiments possess a reaction standard free energy ΔG°_{r×n} within −4 kcal/mol to +4 kcal/mol, the standard free energy determined by Expression (ΔG°₉₋₄−ΔG°₁₂₋₁₃+(ΔG°₈₋₅−ΔG°₃₋₅)) is not between −4 kcal/mol and +4 kcal/mol, and the standard free energy determined by Expression (ΔG°₉₋₄−ΔG°₁₂₋₁₃+(ΔG°₈₋₅−ΔG°₃₋₅)+Rτ ln([BP]/[BPCA]) is not between −5 kcal/mol and +5 kcal/mol. Note that the energy term is defined to be zero if the denoted regions do not exist.

In any of the above embodiments, the probe of the present invention may further comprise a functionalized chemical moiety is conjugated to the 5′ end, to the 3′ end, or to an internal nucleotide of the first universal strand and may also comprise the same on the second universal strand. For example, a fluorophore moiety is conjugated to the first universal strand and a fluorescence quenching moiety is conjugated to the second universal strand. In one embodiment, the fluorophore is within 4 nm of a fluorescence quenching moiety. Additionally, the first universal strand and second universal strand may comprise multiple fluorophore moieties and multiple fluorescence quenching moieties, respectively, wherein the fluorophore is within 4 nm of a quenching moiety. In an alternative embodiment, one or more fluorophore moieties may be conjugated to the second universal strand and one or more quenching moieties may be conjugated to the first universal strand and wherein, the one or more fluorophores may be within 4 nm of the one or more quenching moieties.

In any of the above embodiments, the probe may further comprise any combination of the group consisting of: at least one extra single stranded region between the second region and the third region; between the fifth region and the sixth region; between the tenth region and the first region; between the seventh region and the eleventh region; between the second region and the twelfth region; and between the thirteenth region and the sixth region.

In any of the above embodiments, the probe may comprise a stoichiometric ratio of the protector component (the second universal strand and the protector strand) to probe (comprising protector component and target binding component—first universal strand and complement strand) from about 0.001:1 to about 1000:1.

In one embodiment, a probe is provided comprising a first universal strand, a second universal strand, a protector strand, a complement strand, and at least one auxiliary nucleic acid strand, wherein the second universal strand is indirectly colocalized to the protector strand by the at least one auxiliary nucleic acid strand. In one embodiment, the sequences of the auxiliary nucleic acid strand are arbitrary and do not depend on the sequence of the target.

In another embodiment, a probe is provided comprising a first universal strand, a second universal strand, a protector strand, a complement strand, and at least one auxiliary nucleic acid strand, wherein the first universal strand is indirectly colocalized to the complement strand by the at least one auxiliary nucleic acid strand. In one embodiment, the sequences of the auxiliary nucleic acid strand are arbitrary and do not depend on the sequence of the target.

In any of the above embodiments, any one of the strands of the probe may further comprise a synthetic nucleic acid analog such as LNA, PNA, 2′O-methyl substituted RNA, L-DNA, and speigelmers. In an alternative, any one of the strands of the probe may further comprise synthetic or natural analogs such as isosine, methylated nucleotides, iso-cytosine and iso-guanine, spiegelmer nucleotides, and xDNA.

A process for preparing any of the above embodiments of the present probe is provided. In one embodiment, the first universal strand (A), the second universal strand (B), the protector strand (P), and the complement strand (C) are mixed together in aqueous solution. In one embodiment, the concentration of the complement strand is in excess of the first universal strand, the concentration of the protector strand is in excess of the complement strand, and the concentration of the second universal strand is in excess of the protector strand such that a probe mixture is formed comprising complex BPCA, complex BPC, complex BP, and strand B. In another embodiment, the concentration of the protector strand is in excess of the second universal strand, the concentration of the complement strand is in excess of the protector strand, and the concentration of the first universal strand is in excess of the specific complement strand such that a probe mixture formed comprises complex BPCA, complex PCA, complex CA, and strand A.

In any of the above embodiments, the probe components are thermally annealed following mixing. In one embodiment, the thermal annealing includes heating the mixture to a temperature no less than 65° C., and cooling to a temperature no higher than 45° C. In another embodiment, the thermal annealing includes heating the mixture to a temperature no less than 80° C., and cooling to a temperature no higher than 60° C. In yet another embodiment, the thermal annealing includes heating the mixture to a temperature no less than 95° C., and cooling to a temperature no higher than 75° C.

In an alternative embodiment, the probe components are isothermally annealed through addition of salt or high salinity solutions. In yet another embodiment, the probe components are isothermally annealed through removal or dilution of formamide or other denaturants.

The probes of the present disclosure can be used in a variety of assays including, but not limited to the following: specific DNA or RNA detection or quantitation via fluorescence; specific DNA or RNA imaging via fluorescence; specific DNA or RNA detection, quantitation, or imaging via chromagenic methods (e.g. haptenated probes, and subsequent antibody-based recruitment of horseradish peroxidase (HRP) or alkaline phosphate (AP)); real-time DNA or RNA quantitation, such as through quantitative PCR; and DNA or RNA capture via biotin-streptavidin, gold-thiol, click chemistry, or other chemistries.

Thus, in yet another embodiment, a method for detecting a sequence variance in a sample of nucleic acid is provided. The method comprises introducing a probe of the present disclosure into a sample comprising nucleic acid. The probe can be any probe that has been described herein comprising a complement oligonucleotide that is specific for the sequence variance that is desired to be detected. The “introducing” step should be understood to comprise any method known in the art for administering a nucleic acid probe to a sample containing nucleic acid. The method may further comprise thermal annealing steps as described above prior to the introducing step in order to permit the various oligonucleotide components of the probe to anneal. Additionally, there may be additional thermal cycling steps following the introduction of the probe to the sample in accordance with polymerase chain reaction or other nucleic acid hybridization protocols for causing the probe to base pair with the target. Finally, in the instance a label such as a fluorophore is conjugated to a universal oligonucleotide of the probe, the method further comprises either imaging the sample to detect the fluorophore signal or collecting data based on the level of fluorescence produced as a result of the probes interaction with the target.

All regions described above (first region, second region, third region, etc.) should be understood to reference the particular region of the particular strand as described above throughout this disclosure. For example, “first region” as used herein should be understood to refer to the above described region of the second universal strand throughout this disclosure.

Referring now to FIG. 2, The X-probe's intended function is to hybridize to a specific Target nucleic acid sequence or subsequence T, such that after hybridization, Target T is colocalized to the first universal strand A via the complement strand C (which is coupled to the first universal strand A via hybridization of at least region 6 to region 7). Target T comprises regions 8 and 9, such that region 8 is partially or fully complementary to region 5, and region 9 is partially or fully complementary to region 4. Importantly, regions 8 and 9 do not have any necessary patterns of interaction with regions 7 or 11, the regions on the first universal strand.

Thus, the ability to colocalize target T to a functionalized oligonucleotide A of completely independent sequence has important economic implications, as the same oligonucleotide A can be mass-produced for function with any or many different sequences of T via the specificity provided by the specific complementary strand. Many different chemical moieties could potentially be functionalized to first universal strand A—for example, a fluorophore, a biotin, a digoxigenin or other hapten, a thiol, a N-hydroxysuccinimide group, or an alkyne or azide group.

Referring now to FIG. 3, one embodiment of the present disclosure provides a probe that provides conditional fluorescence such that fluorescence increases upon hybridization of the X-probe to its intended target T. In this embodiment, the first universal strand A is functionalized with a fluorophore at the 3′ end, and the second universal strand B is functionalized with a fluorescence quenching moiety (such as a Black Hole Quencher or an Iowa Black Quencher) at the 5′ end. The fluorophore on A is initially within 4 nm of the quenching moiety on B, and fluorescence is low due to contact, Dexter, or Forster quenching. Upon hybridization of T, the complex containing B and its quencher dissociates from the complex containing A and its fluorophore, and fluorescence increases. Thus, fluorescence increase can represent the detection of a particular nucleic acid sequence, and furthermore may be used to infer the concentration of said nucleic acid sequence.

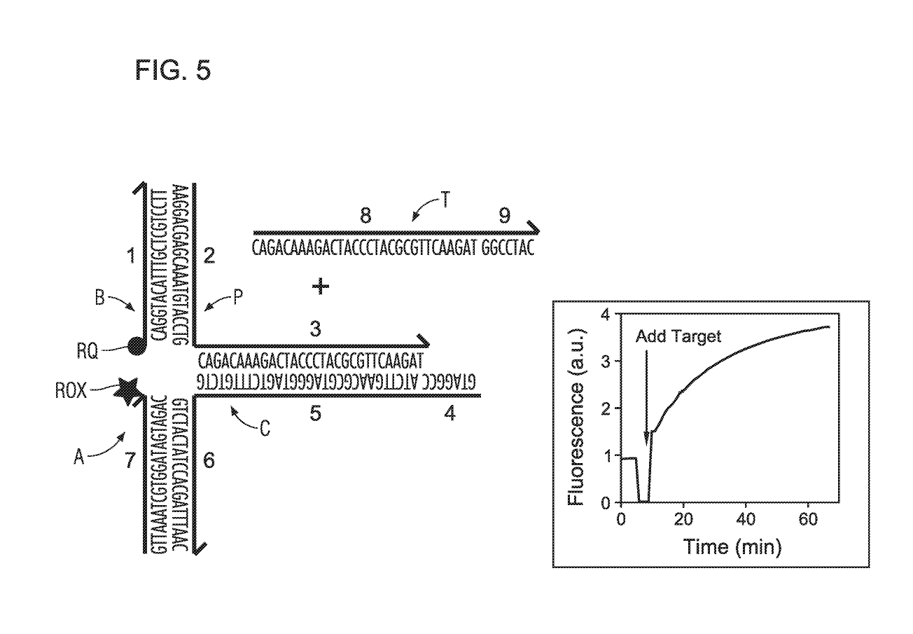

Referring now to FIG. 4, on example of preparing a probe of the present disclosure is provided. In this example, different amounts of the X-probe component strands are mixed in the ratio of 5 to 3 and 1.5 to 1, of strands B to P and C to A, respectively, in an aqueous solution of 0.75 M NaCl. Subsequently, the mixture is thermally annealed via heating to 95° C., and cooling to 25° C. uniformly over a period of 70 minutes. The X-probe mixture will then contain the X-probe BPCA, as well as products BPC, BP, and B. The other products may serve important functional roles, such as AP in the case of high specificity X-probes. The X-probe mixture is then ready for use.

In this example preparation process, the stoichiometry (5:3:1.5:1) was selected, but it should be understood that many different stoichiometries could be used. In this example preparation process, the salinity of the buffer (0.75 M Na+) was selected, but an aqueous solution with no less than 50 mM of a monovalent cation (such as Na+) or 1 mM of a divalent cation (such as Mg2+) could be used. In this example preparation process, the thermal annealing protocol was selected, but various different heating and cooling profiles may be used. Alternatively, thermal annealing may be skipped altogether and the components could be added to a sample independently.

High target-binding specificity of the probe of the present disclosure is achieved at equilibrium when the reaction standard free energy (ΔG°_{r×n}) is within −4 and +4 kcal/mol, in entropically balanced reactions with equal numbers of reactant and product species. The reaction standard free energy ΔG°_{r×n} can be calculated by summing a number of ΔG° terms corresponding to the thermodynamics of different parts of the X-probe (e.g. binding between complementary regions), as follows:

ΔG°_{r×n}=ΔG°₉₋₄−ΔG°₁₀₋₁₁−ΔG°₁₂₋₁₃−ΔG°_{ML}+(ΔG°₈₋₅−ΔG°₃₋₅)−ΔG°_{label}

In the above equation, the energy terms with subscripted by numbers represent the standard free energy of the hybridization between the two referenced regions. For example, the term ΔG°₉₋₄ represents the standard free energy of the hybridization between the ninth region (in target T) and the fourth region in the complement strand of the present probe. The term ΔG°_{ML} represents the standard free energy of the multi-loop formed at the junction of the four paired helixes. The term ΔG°_{label} represents the standard free energy difference between the thermodynamic contribution of the labels on the first universal strand when they in close proximity of the second universal strand versus when they are delocalized. It should be noticed that the energy term is defined to be zero if the denoted regions do not exist.

The standard free energy of the hybridization between regions of the various strands of the present probe is calculated by using the nearest neighbor model as described in Santa Lucia, J. & Hicks, D. The thermodynamics of DNA structural motifs. Annu Rev Biophys Biomol Struct 33, 415-440 (2004) for DNA and Sugimoto, N. et al. Thermodynamic parameters to predict stability of RNA/DNA hybrid duplexes. Biochemistry 34, 11211-11216 (1995) for RNA. In this method, two adjacent base pairs comprise one stack, with reported enthalpy (ΔH°) and entropy (ΔS°) values for DNA and RNA. The standard free energy of each stack at temperature τ is calculated as ΔG°=ΔH°−τΔS°. The standard free energy of a hybridized helix can be evaluated by summing all the stack energies.

Specifically, ΔG°₉₋₄ comprises the standard free energy of all the stacks in duplex 9-4 (i.e. in the ninth (9) region of the target nucleic acid hybridized to the fourth (4) region of the specific complement strand), the standard free energy of the neighboring stack at the junction between duplex 9-4 and duplex 3-5 (third and fifth regions), and the thermodynamic penalty of hybridization initiation (ΔG°ᵢₙᵢₜ), due to the entropy loss of orienting two nucleic acid molecules for hybridization. The value of ΔG°ᵢₙᵢₜ can be calculated based on the published parameters of ΔH°ᵢₙᵢₜ and ΔS°ᵢₙᵢₜ.

ΔG°₁₀₋₁₁ comprises the standard free energy of all the stacks in duplex 10-11 (tenth region and eleventh region).

ΔG°₁₂₋₁₃ comprises the standard free energy of all the stacks in duplex 12-13 (twelfth region and thirteenth region) and the standard free energy of the neighboring stack at the junction between duplex 12-13 and duplex 3-5.

ΔG°₈₋₅ comprises the standard free energy of all the stacks in duplex 8-5.

ΔG°₃₋₅ comprises the standard free energy of all the stacks in duplex 3-5 and the thermodynamic penalty of hybridization initiation (ΔG°ᵢₙᵢₜ).

The standard free energy of the multi-loop ΔG°_{ML} can be evaluated by published online software such as that described in Zuker, M. Mfold web server for nucleic acid folding and hybridization prediction. Nucleic Acids Res. 31, 3406-3415 (2003) (mfold). For example, and the thermodynamic effect of multi-loop composed by the exact sequences as shown in our examples is about +4.5 kcal/mol (37° C., 0.75 M Na⁺, calculated by mfold).

It has been known that the functionalized groups can have significant thermodynamic effects, stabilizing or destabilizing nucleic acid hybridization. Although the energy ΔG°_{label} cannot be calculated precisely, it can be experimentally characterized. For example, we observed the thermodynamic effect of interaction between the ROX fluorophore and the RQ quencher is between −3 to −4 kcal/mol (37° C., 0.75 M Na⁺).

We have previously shown in PCT application number PCT/US14/52827, the contents of which are incorporated herein by reference, that the probe behavior can be fine-tuned post-synthesis by tuning the ratio of the concentration of the various components of the probe in solution. Furthermore, for more basic explanation on the interaction between the target and probes comprising a complex of a complement strand and a protector strand, see published international application no. WO2012058488, which is incorporated by reference herein in its entirety.

As applied to our current invention, a good tradeoff between specificity and sensitivity is achieved when the standard free energy determined by Expression 1 (ΔG°_{r×n}) is within 5 kcal/mol of the standard free energy determined by Expression −Rτ ln([BP]/[BPCA]) wherein R is the ideal gas constant, τ is the temperature in Kelvin, [BP] is the final concentration of the complex BP in solution, and [BPCA] is the final concentration of the X-probe in solution.

In practice, the concentration of BP can be rationally determined such that the complex BP is in excess of the complex BPCA to improve the specificity of the X-probe. Furthermore, the lengths and sequences of the regions in the X-probe component oligonucleotides can be rationally designed to achieve ΔG°_{r×n} within −4 kcal/mol and +4 kcal/mol.

When the highly specific X-probe is designed to be conditionally fluorescent, additional design criteria of fluorescence quenching ratio will impact the design of the optional regions 10, 11, 12, and 13.

In general, to ensure that the complex BP can be fully displaced by the target molecule in the operational temperatures and salinity conditions, the length of the optional regions need to be short enough such that the first universal strand and the second universal strand cannot form stable partially double-stranded complex via the hybridization between region 10 and region 11. However, if the lengths of the region 10 and region 11 are too short, the fluorophore and the quenching moiety might not be within 4 nm from each other, increasing the fluorescence background. Therefore, the base pairs of optional regions need to be distributed according to the operation temperatures and salinity condition. In the examples described below, the length of regions 10 and 11 were selected to be 7 nucleotides, in consideration of the operational temperature of 37° C. and operational salinity of 0.75 M Na⁺.

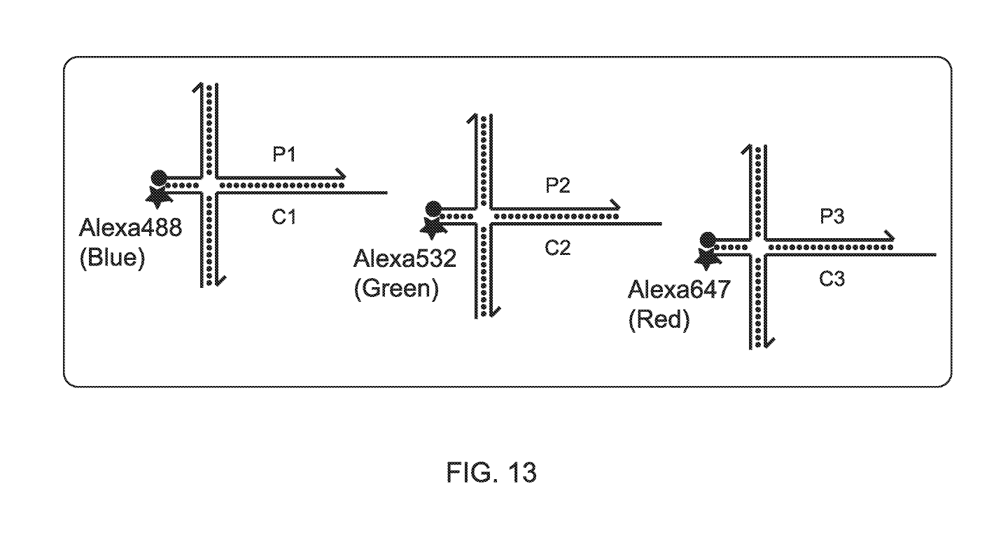

In addition to use of an individual X-probe for detection of a single target sequence, potentially in a background of other nucleic acids with similar sequence, multiple X-probes can simultaneously exist in solution and be used for parallel detection of multiple analyte sequences as provided in FIG. 12. For example, a mixture of multiple conditionally fluorescent X-probes can react with any or all of a number of different target sequences to yield increased fluorescence of the same spectral properties (a.k.a. color). The process of preparing multiple X-probes for simultaneous function generally comprises combining sufficient quantities of the first universal strand and second universal strand with the appropriate amounts of different protector strands and complement strands. These can then be optionally thermally annealed in a “one-pot” protocol.

The ability to easily construct pools of X-probes to simultaneously probe multiple target nucleic acid sequences has important implications for clinical diagnostics. For example, there are many known cancer mutations such as those reported in Forbes, S. et al. COSMIC 2005. Br. J. Cancer 94, 318-322 (2006), and any particular cancer patient will only have a small subset of the known mutations. Thus, X-probes against known mutations can be pooled together, such that any significant fluorescence response will indicate the presence of at least one cancer mutation, and by implication existence or increased risk of tumors in the body. As another example, certain strains of infectious diseases possess different antibiotics resistances, and these resistances can be conferred via mutations on different genes; parallel X-probe detection of mutations in any antibiotics resistance genes can help enable accurate infectious disease diagnostics and profiling.

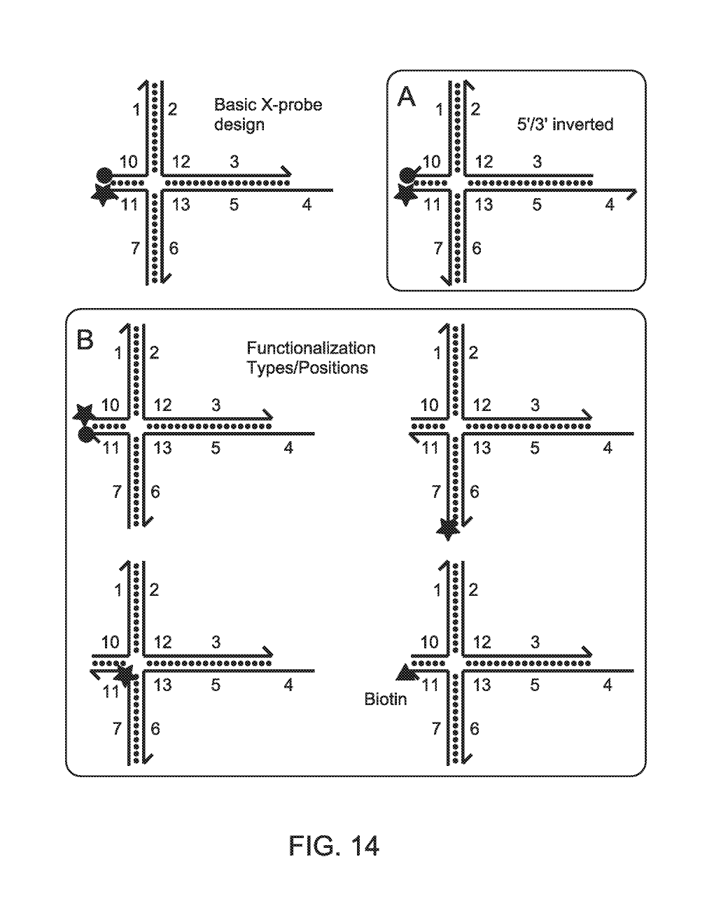

In an alternative embodiment, multiplexed detection of different targets is provided. Referring now to FIG. 13, in contrast to parallel detection, multiplexed X-probe systems are functionalized with several different moieties, and produce different results based on the identity of the target sequence(s) present. For example, multiplexed conditionally fluorescent X-probes may be labeled with several different fluorophores that are spectrally distinct (FIG. 13). For applications such as fluorescence in situ hybridization, the ability to simultaneously detect and localize multiple targets can be useful when analyte samples are limited (e.g. drawn from patient biopsy).

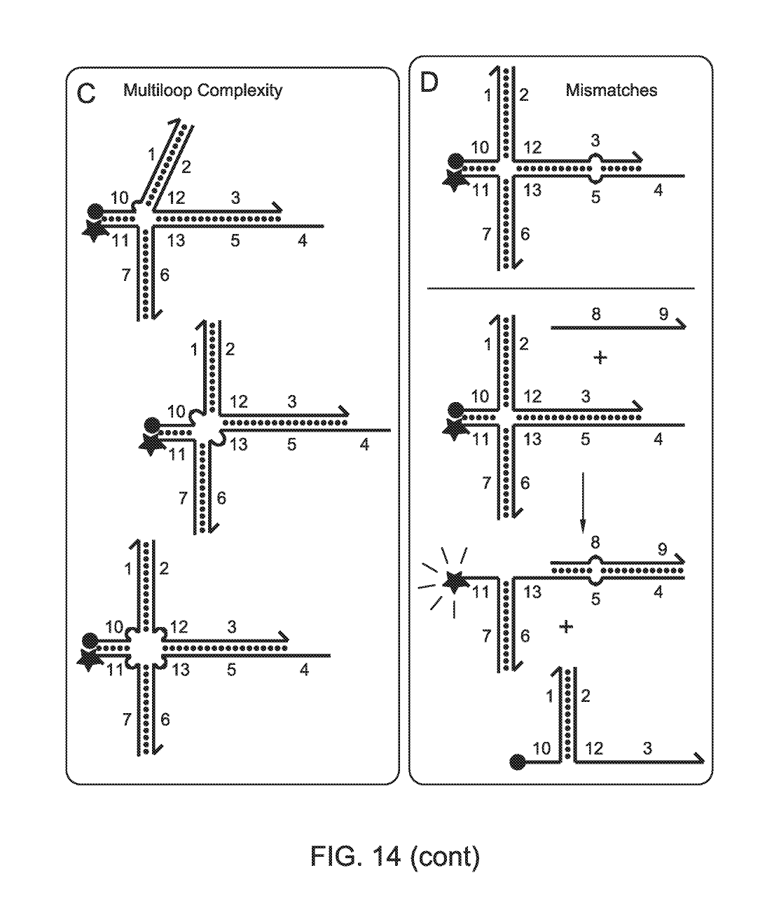

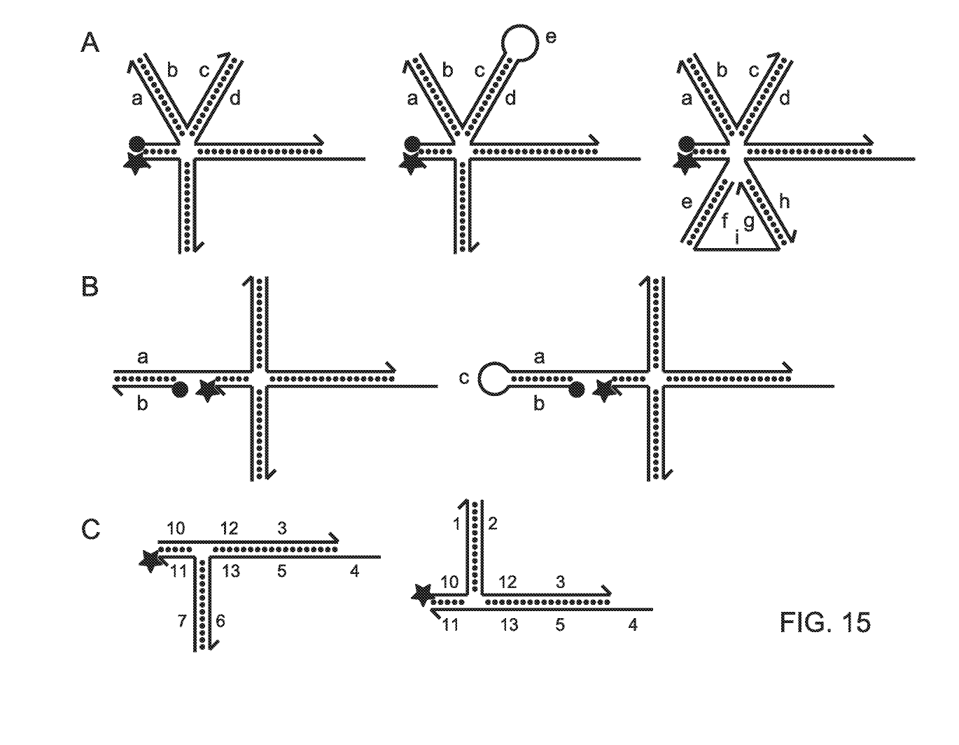

One advantage of the present probe system is the concept of decoupling the sequence of the functionalized strand from the sequence of the target. Many different variants of the basic X-probe design should function similarly. These variants are provided in FIGS. 14 and 15 with the latter including significant changes in either the X-probe component strands' regions or addition of new regions or strands.

Referring now to FIG. 14, variations include the 5′/3′ orientation of the X-probe (panel A), the type and position of functionalization (panel B), the addition of additional single-stranded nucleotides between regions (panel C), and region sequences that imperfectly hybridize to their intended complements (e.g. imperfect binding between regions 3 and 5, or between regions 8 and 5). In each of these cases, the number of component strands in the X-probe is the same, and the overall behavior of the X-probe variant can be roughly predicted based on the thermodynamics of nucleic acid hybridization motifs.

Referring now to FIG. 15, examples of more complex variations are described. Specifically, additional helices are introduced in the X-probe design, so that the X-probe contains 5 or more duplex DNA “arms” (the basic X-probe design comprises 3 or 4 duplex arms). These designs similarly achieve decoupling of the sequence of functionalized strands from that of the target sequence, albeit with extra cost of additional nucleotide bases or oligonucleotides. Introduction of additional auxiliary strands or regions does not change the mechanism of action of the X-probe, and are covered by this invention.

Alternatively, the morphology of the first universal strand A or second universal strand B may differ from embodiments described earlier, for example by incorporating additional regions or strands as shown in (panel B) of FIG. 15.

Finally, a number of reduced designs also function in the spirit of the X-probes by removing one or more of the regions and strands, in the case where only the first universal strand is functionalized, and not the second universal strand (panel C).

### Examples

### Example 1

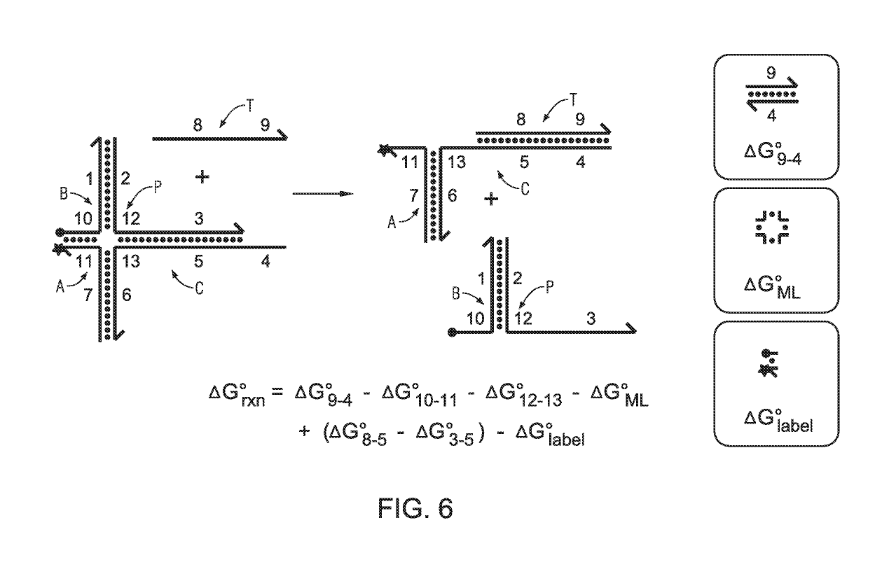

In this example, a conditionally-fluorescent X-probe of the present disclosure is used to identify the presence of a target nucleic acid in a sample. Referring now to FIG. 5, the probe of the present example comprises the following: universal strand A (region 7) has SEQ ID. NO. 2 with a functional ROX group conjugated to the 3′ end, universal strand B (region 1) has SEQ ID. NO. 3 with a quencher RQ conjugated to the 5′ end, protector strand P (regions 2 and 3) has SEQ ID. NO. 4, and complementary strand (regions 4, 5, and 6) has SEQ ID. NO. 5. The target nucleic acid of the present example comprises SEQ ID NO. 1.

The experiment was conducted in 1200 μL cuvette with appropriate buffer (typically 5×PBS). X-probe solution is incubated in the cuvette in Horiba Fluoromax-4 machine for 20 minutes to allow temperature equilibration before the start of data acquisition. The buffer and X-probe are further incubated in the cuvette for an additional 5 to 20 minutes after fluorescence data acquisition begins in order to establish the background fluorescence. Subsequently, the cuvettes are removed from the machine and target solutions (short synthetic oligonucleotide depicted as target T of the FIG. 5) are added to the cuvette. The cuvette is then capped with a fitted Teflon stopper, and the solution is mixed by inverting the cuvette roughly 10 times. During this reagent addition and mixing process, the data acquisition continues, resulting in a low fluorescence level that is indicative of the cuvettes being removed from the machine. Upon completion of mixing, the cuvettes are replaced into the machine.

As demonstrated in the inset of FIG. 5, the application of nucleic acid comprising target T to a sample containing this exemplary X-probe results in significantly increases in fluorescence thereby demonstrating the specificity of the probe to the target as well as the conditional fluorescence.

### Example 2

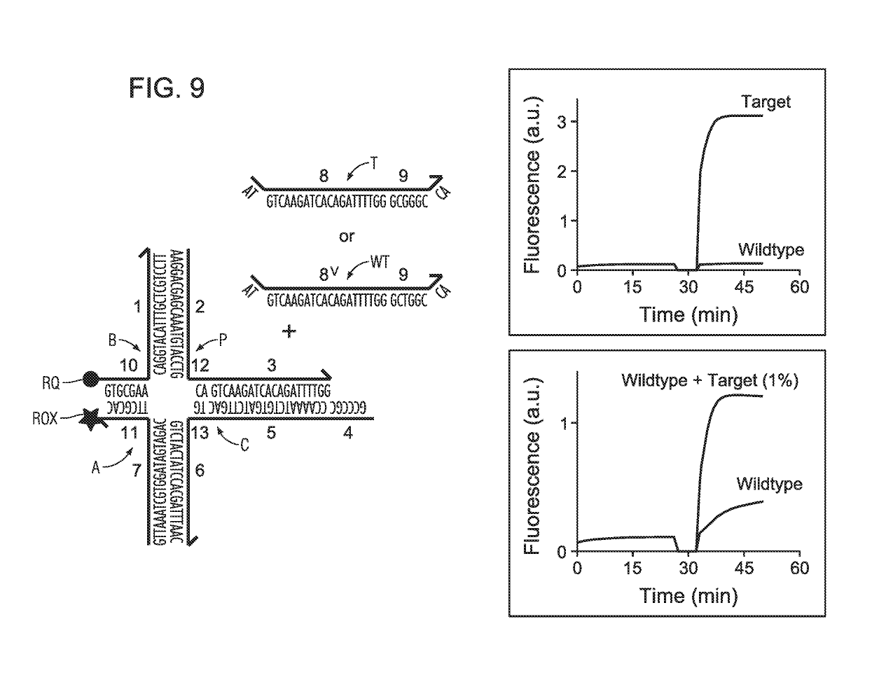

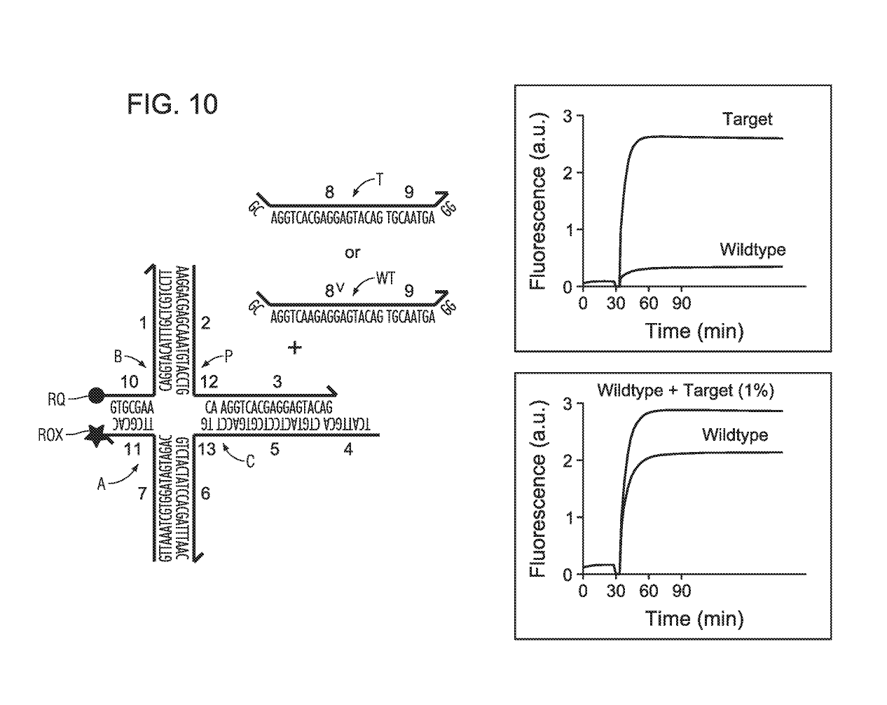

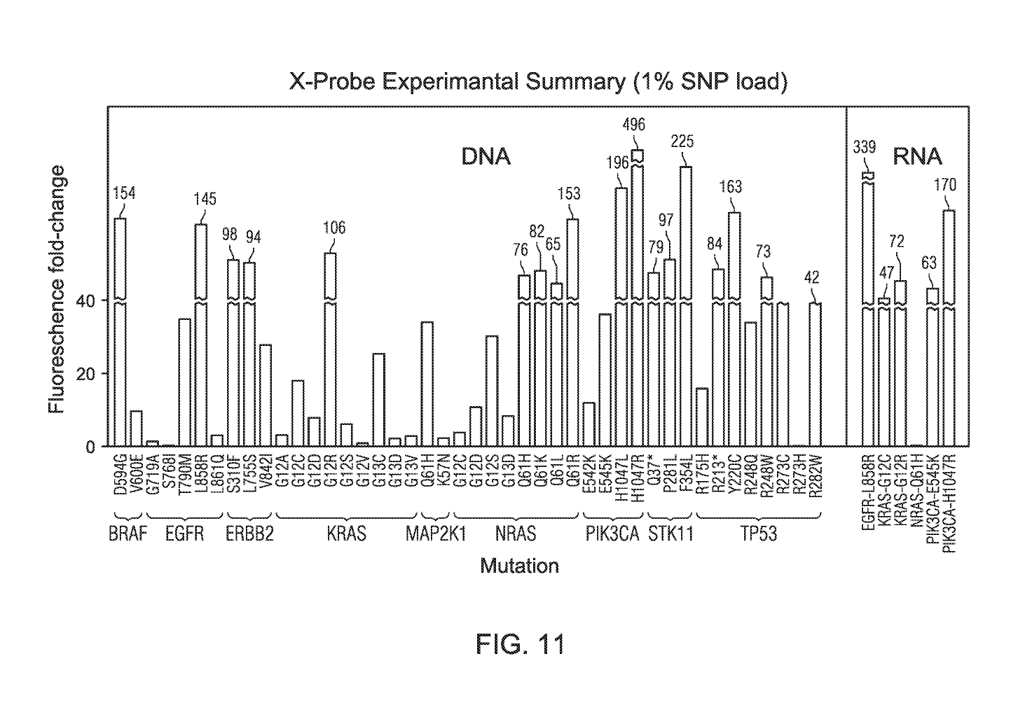

In this example, three different conditionally-fluorescent X-probes of the present disclosure (with common universal strands A and B) are used to identify the presence of three different target nucleic acids (sequence for TP53 carrying the R273C (c.817C>T) mutation—FIG. 8; sequence for EGFR carrying the L858R, (c.2373T>G) mutation—FIG. 9; and sequence for KRAS carrying the Q61H, (c.183A>C) mutation) in a sample also containing a nucleic acid carrying the wild type sequence corresponding to each described mutation. This mutation was taken from the COSMIC database which provides single-base mutations commonly found in cancer patients. FIGS. 8-10 provides the structure and sequence of the probe used in this example and the sequences of the target (mutated) and wild type nucleic acids. The experimental design for this example is the same as described above in Example 1. The experimental results panels on the right side of each figure shows both the fluorescence response of an equal amount of target and wild type (top) and the additional fluorescence response of a 1% load of target in a background of wild type.

As can be seen in the panels on FIGS. 8-10, the X-probe efficiently discriminates the single-base change by binding much more significantly to its intended target. More specifically, even in the instances where the target constitutes only 1% of the total nucleic acid of the sample (where the remaining is the wild type), the probes of the present disclosure are able to discriminate and bind preferentially to the target.

### Example 3

In addition to the examples described above and depicted in FIGS. 5, and 8-10, X-probes were also designed and tested for 41 other cancer mutations using the same experimental design described above with respect to Examples 1 and 2 (note the results for the probes and targets in Examples 1 and 2 are included in this example such that there is a total of 44 mutations screened using the probes of the present disclosure). The sequences for the universal strands are provided in Table 1. The sequences for the protector strand P and complement strand C of the probe as well as the target nucleic acid sequence carrying the mutation (SNV), and the wild type (WT) sequence for each mutation are provided in Tables 2-10. RNA sequences for mutant (SNV) and wild type (WT) are the analogs of the DNA sequences (with T's replaced by U's) and are not explicitly shown. In functionalized sequences, /3Rox_N/ denotes the IDT (Integrated DNA Technologies) entry code for the 3′ ROX (carboxy-X-rhodamine) fluorophore functionalized by NHS ester chemistry, and /5IAbRQ/ the IDT entry code for the 5′ Iowa Black Red Quencher group.

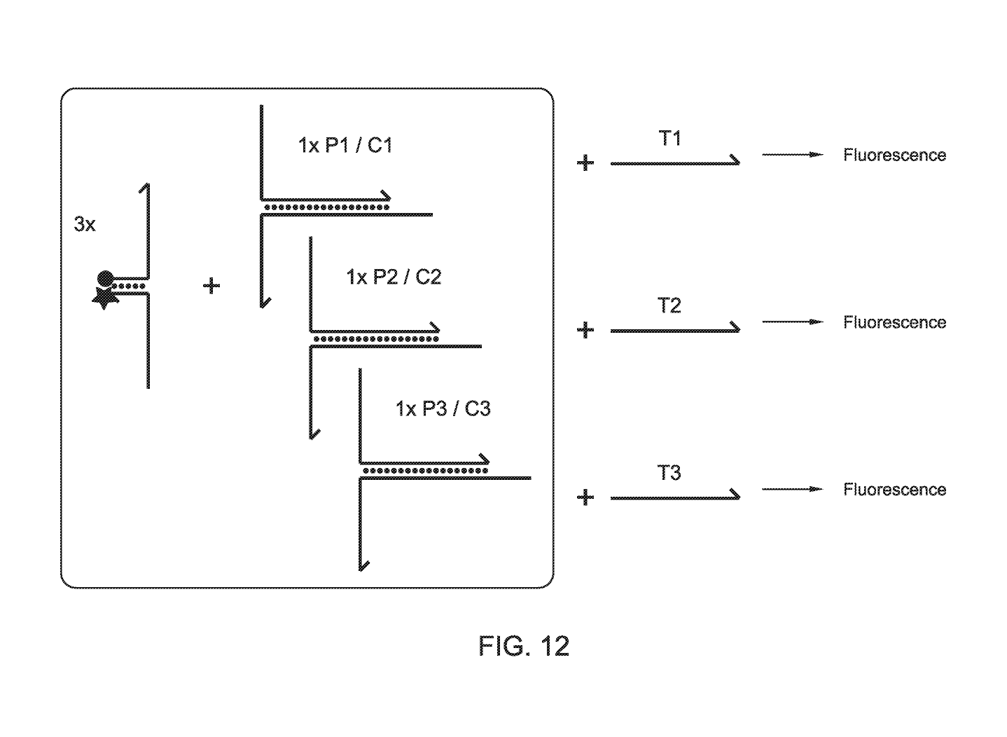

These experimental results are summarized in FIG. 11. As can be seen, the X-probes are able to consistently detect a 1% cancer mutation load in a background of synthetic oligonucleotides carrying the wild type sequence.

TABLE 1
Sequences of universal X-Probe strands.

| Length of | | SEQ | |
|---|---|---|---|
| regions 10 | Universal | ID | |
| and 11 (nt) | Strand | NO. | Sequence |
| 7 | A | 13 | GTTAAATCGTGGATAGTAGAC TTCGCAC/3Rox N/ |
| | B | 14 | /5IAbRQ/GTGCGAACAGGTACATTTGCTCGTCCTT |

TABLE 2
Sequences of SNV, WT, and Probe protector (P) and complement (C)
strands for BRAF mutations.

| | | SEQ | |
|---|---|---|---|
| | | ID | |
| Mutation | Species | NO. | Sequence |
| BRAF- | SNV | 27 | ATAGGTGGTTTTGGTCTAGCTACAGTGAAA |
| D594G | WT | 28 | ATAGGTGATTTTGGTCTAGCTACAGTGAAA |
| (c.1781 | P | 29 | AAGGACGAGCAAATGTACCTGCAAGGTGGTTTTGGTCTA |
| A > G) | | | GC |
| | C | 30 | TTCACTGTAGCTAGACCAAAACCACCTTGGTCTACTATCC |
| | | | ACGATTTAAC |
| BRAF- | SNV | 31 | ATAGGTGATTTTGGTCTAGCTACAGAGAAA |
| V600E | WT | 28 | ATAGGTGATTTTGGTCTAGCTACAGTGAAA |
| (c.1799 | P | 32 | AAGGACGAGCAAATGTACCTGCAAGGTGATTTTGGTCTA |
| T > A) | | | G |
| | C | 33 | TCTCTGTAGCTAGACCAAAATCACCTTGGTCTACTATCCA |
| | | | CGATTTAAC |

TABLE 3
Sequences of SNV, WT, and target-dependent X-Probe strands for
EGFR mutations.

| | | SEQ | |
|---|---|---|---|
| | | ID | |
| Mutation | Species | NO. | Sequence |
| EGFR- | SNV | 34 | TTCAAAAAGATCAAAGTGCTGGCCTCCGGT |
| G719A | WT | 35 | TTCAAAAAGATCAAAGTGCTGGGCTCCGGT |
| (c.2156 | P | 36 | AAGGACGAGCAAATGTACCTGCACAAAAAGATCAAAGTG |
| G > C) | | | CTGG |
| | C | 37 | CGGAGGCCAGCACTTTGATCTTTTTGTGGTCTACTATCCA |
| | | | CGATTTAAC |
| EGFR- | SNV | 38 | GCCTACGTGATGGCCATCGTGGACAACCCC |
| S768I | WT | 39 | GCCTACGTGATGGCCAGCGTGGACAACCCC |
| (c.2303 | P | 40 | GGTTGTCCACGATGGCCATCACGTAGTGGTCTACTATCCA |
| G > T) | | | CGATTTAAC |
| | C | 40 | GGTTGTCCACGATGGCCATCACGTAGTGGTCTACTATCCA |
| | | | CGATTTAAC |
| EGFR- | SNV | 41 | GTGCAGCTCATCATGCAGCTCATGCCCTTC |
| T790M | WT | 42 | GTGCAGCTCATCACGCAGCTCATGCCCTTC |
| (c.2369 | P | 43 | AAGGACGAGCAAATGTACCTGCAGCAGCTCATCATGCAG |
| C > T) | | | CTC |
| | C | 44 | AGGGCATGAGCTGCATGATGAGCTGCTGGTCTACTATCCA |
| | | | CGATTTAAC |
| EGFR- | SNV | 21 | ATGTCAAGATCACAGATTTTGGGCGGGCCA |
| L858R | WT | 22 | ATGTCAAGATCACAGATTTTGGGCTGGCCA |
| (c.2573 | P | 19 | AAGGACGAGCAAATGTACCTGCAGTCAAGATCACAGATT |
| T > G) | | | TTGG |
| | C | 20 | GCCCGCCCAAAATCTGTGATCTTGACTGGTCTACTATCCA |
| | | | CGATTTAAC |
| EGFR- | SNV | 45 | TGGCCAAACAGCTGGGTGCGGAAGAGAAAG |
| L861Q | WT | 46 | TGGCCAAACTGCTGGGTGCGGAAGAGAAAG |
| (c.2582 | P | 47 | AAGGACGAGCAAATGTACCTGCAGCCAAACAGCTGGGTG |
| T > A) | | | CG |
| | C | 48 | TTTCTCTTCCGCACCCAGCTGTTTGGCTGGTCTACTATCCA |
| | | | CGATTTAAC |

TABLE 4
Sequences of SNV, WT, and target-dependent X-Probe strands for
ERBB2 mutations.

| | | SEQ | |
|---|---|---|---|
| | | ID | |
| Mutation | Species | NO. | Sequence |
| ERBB2- | SNV | 49 | ACTACCTTTCTACGGACGTGGGATTCTGCA |
| S310F | WT | 50 | ACTACCTTTCTACGGACGTGGGATCCTGCA |
| (c.929 | P | 51 | AAGGACGAGCAAATGTACCTGCATACCTTTCTACGGACG |
| C > T) | | | TG |
| | C | 52 | CAGAATCCCACGTCCGTAGAAAGGTATGGTCTACTATCCA |
| | | | CGATTTAAC |
| ERBB2- | SNV | 53 | TTCCAGTGGCCATCAAAGTGTCGAGGGAAA |
| L755S | WT | 54 | TTCCAGTGGCCATCAAAGTGTTGAGGGAAA |
| (c.2264 | T | 55 | AAGGACGAGCAAATGTACCTGCACCAGTGGCCATCAAAG |
| P > C) | | | TG |
| | C | 56 | TCCCTCGACACTTTGATGGCCACTGGTGGTCTACTATCCA |
| | | | CGATTTAAC |
| ERBB2- | SNV | 57 | GGATGTGCGGCTCATACACAGGGACTTGGC |
| V8421 | WT | 58 | GGATGTGCGGCTCGTACACAGGGACTTGGC |
| (c.2524 | P | 59 | AAGGACGAGCAAATGTACCTGCAATGTGCGGCTCATACA |
| G > A) | | | CA |
| | C | 60 | CAAGTCCCTGTGTATGAGCCGCACATTGGTCTACTATCCA |
| | | | CGATTTAAC |

TABLE 5
Sequences of SNV, WT, and target-dependent X-Probe strands for
KRAS mutations.

| | | SEQ | |
|---|---|---|---|
| | | ID | |
| Mutation | Species | NO. | Sequence |
| KRAS- | SNV | 61 | CTTGTGGTAGTTGGAGCTGCTGGC |
| G12A | WT | 62 | CTTGTGGTAGTTGGAGCTGGTGGC |
| (c.35G > | P | 63 | AAGGACGAGCAAATGTACCTGCAACTTGTGGTAGTTGGA |
| C) | | | G |
| | C | 64 | GCCAGCAGCTCCAACTACCACAAGTTGGTCTACTATCCAC |
| | | | GATTTAAC |
| KRAS- | SNV | 65 | CTTGTGGTAGTTGGAGCTTGTGGC |
| G12C | WT | 62 | CTTGTGGTAGTTGGAGCTGGTGGC |
| (c.34G > | P | 66 | AAGGACGAGCAAATGTACCTGCAACTTGTGGTAGTTGGA |
| T) | | | GC |
| | C | 67 | GCCACAAGCTCCAACTACCACAAGTTGGTCTACTATCCAC |
| | | | GATTTAAC |
| KRAS- | SNV | 68 | CTTGTGGTAGTTGGAGCTGATGGC |
| G12D | WT | 62 | CTTGTGGTAGTTGGAGCTGGTGGC |
| (c.35G > | P | 63 | AAGGACGAGCAAATGTACCTGCAACTTGTGGTAGTTGGA |
| A) | | | G |
| | C | 69 | GCCATCAGCTCCAACTACCACAAGTTGGTCTACTATCCAC |
| | | | GATTTAAC |
| KRAS- | SNV | 70 | CTTGTGGTAGTTGGAGCTCGTGGC |
| G12R | WT | 62 | CTTGTGGTAGTTGGAGCTGGTGGC |
| (c.34G > | P | 66 | AAGGACGAGCAAATGTACCTGCAACTTGTGGTAGTTGGA |
| C) | | | GC |
| | C | 71 | GCCACGAGCTCCAACTACCACAAGTTGGTCTACTATCCAC |
| | | | GATTTAAC |
| KRAS- | SNV | 72 | CTTGTGGTAGTTGGAGCTAGTGGC |
| G12S | WT | 62 | CTTGTGGTAGTTGGAGCTGGTGGC |
| (c.34G > | P | 63 | AAGGACGAGCAAATGTACCTGCAACTTGTGGTAGTTGGA |
| A) | | | G |
| | C | 73 | GCCACTAGCTCCAACTACCACAAGTTGGTCTACTATCCAC |
| | | | GATTTAAC |
| KRAS- | SNV | 74 | CTTGTGGTAGTTGGAGCTGTTGGC |
| G12V | WT | 62 | CTTGTGGTAGTTGGAGCTGGTGGC |
| (c.35G > | P | 63 | AAGGACGAGCAAATGTACCTGCAACTTGTGGTAGTTGGA |
| T) | | | G |
| | C | 75 | GCCAACAGCTCCAACTACCACAAGTTGGTCTACTATCCAC |
| | | | GATTTAAC |
| KRAS- | SNV | 76 | CTTGTGGTAGTTGGAGCTGGTTGC |
| G13C | WT | 62 | CTTGTGGTAGTTGGAGCTGGTGGC |
| (c.37G > | P | 63 | AAGGACGAGCAAATGTACCTGCAACTTGTGGTAGTTGGA |
| T) | | | G |
| | C | 77 | GCAACCAGCTCCAACTACCACAAGTTGGTCTACTATCCAC |
| | | | GATTTAAC |
| KRAS- | SNV | 78 | CTTGTGGTAGTTGGAGCTGGTGACGTAGGC |
| G13D | WT | 79 | CTTGTGGTAGTTGGAGCTGGTGGCGTAGGC |
| (c.38G > | P | 80 | AAGGACGAGCAAATGTACCTGCATGTGGTAGTTGGAGCT |
| A) | | | GG |
| | C | 81 | CTACGTCACCAGCTCCAACTACCACATGGTCTACTATCCA |
| | | | CGATTTAAC |
| KRAS- | SNV | 82 | CTTGTGGTAGTTGGAGCTGGTGTCGTAGGC |
| G13V | WT | 79 | CTTGTGGTAGTTGGAGCTGGTGGCGTAGGC |
| (c.38G > | P | 80 | AAGGACGAGCAAATGTACCTGCATGTGGTAGTTGGAGCT |
| T) | | | GG |
| | C | 83 | CTACGACACCAGCTCCAACTACCACATGGTCTACTATCCA |
| | | | CGATTTAAC |
| KRAS- | SNV | 25 | GCAGGTCACGAGGAGTACAGTGCAATGAGG |
| Q61H | WT | 26 | GCAGGTCAAGAGGAGTACAGTGCAATGAGG |
| (c.183A > | P | 23 | AAGGACGAGCAAATGTACCTGCAAGGTCACGAGGAGTAC |
| C) | | | AG |
| | C | 24 | TCATTGCACTGTACTCCTCGTGACCTTGGTCTACTATCCAC |
| | | | GATTTAAC |

TABLE 6
Sequences of SNV, WT, and target-dependent X-Probe strands for
MAP2K1 mutation.

| | | SEQ | |
|---|---|---|---|
| | | ID | |
| Mutation | Species | NO. | Sequence |
| MAP2K | SNV | 84 | ACCCAGAATCAGAAGGTGGGAGAACTGAAG |
| 1-K57N | WT | 85 | ACCCAGAAGCAGAAGGTGGGAGAACTGAAG |
| (c.171G > | P | 86 | AAGGACGAGCAAATGTACCTGCACCAGAATCAGAAGGTG |
| T) | | | GG |
| | C | 87 | TTCAGTTCTCCCACCTTCTGATTCTGGTGGTCTACTATCCA |
| | | | CGATTTAAC |

TABLE 7
Sequences of SNV, WT, and target-dependent X-Probe strands for NRAS
mutations

| | | SEQ | |
|---|---|---|---|
| | | ID | |
| Mutation | Species | NO. | Sequence |
| NRAS- | SNV | 88 | GTGGTTGGAGCATGTGGTGTTGGGAAAAGC |
| G12C | WT | 89 | GTGGTTGGAGCAGGTGGTGTTGGGAAAAGC |
| (c.34G > | P | 90 | AAGGACGAGCAAATGTACCTGCAGGTTGGAGCATGTGGT |
| T) | | | GTT |
| | C | 91 | CTTTTCCCAACACCACATGCTCCAACCTGGTCTACTATCC |
| | | | ACGATTTAAC |
| NRAS- | SNV | 92 | GTGGTTGGAGCAGATGGTGTTGGGAAAAGC |
| G12D | WT | 89 | GTGGTTGGAGCAGGTGGTGTTGGGAAAAGC |
| (c.35G > | P | 93 | AAGGACGAGCAAATGTACCTGCAGGTTGGAGCAGATGGT |
| A) | | | GTT |
| | C | 94 | CTTTTCCCAACACCATCTGCTCCAACCTGGTCTACTATCC |
| | | | ACGATTTAAC |
| NRAS- | SNV | 95 | TACAAACTGGTGGTGGTTGGAGCAAGTGGT |
| G12S | WT | 96 | TACAAACTGGTGGTGGTTGGAGCAGGTGGT |
| (c.34G > | P | 97 | AAGGACGAGCAAATGTACCTGCTCAAACTGGTGGTGGTT |
| A) | | | GGA |
| | C | 98 | CACTTGCTCCAACCACCACCAGTTTGAG |
| | | | GTCTACTATCCACGATTTAAC |
| NRAS- | SNV | 99 | GTGGTTGGAGCAGGTGATGTTGGGAAAAGC |
| G13D | WT | 89 | GTGGTTGGAGCAGGTGGTGTTGGGAAAAGC |
| (c.38G > | P | 100 | AAGGACGAGCAAATGTACCTGCAGGTTGGAGCAGGTGAT |
| A) | | | GTT |
| | C | 101 | CTTTTCCCAACATCACCTGCTCCAACCTGGTCTACTATCC |
| | | | ACGATTTAAC |
| NRAS- | SNV | 102 | ATACTGGATACAGCTGGACATGAAGAGTAC |
| Q61H | WT | 103 | ATACTGGATACAGCTGGACAAGAAGAGTAC |
| (c.183A > | P | 104 | AAGGACGAGCAAATGTACCTGCAACTGGATACAGCTGGA |
| T) | | | C |
| | C | 105 | ACTCTTCATGTCCAGCTGTATCCAGTTGGTCTACTATCCA |
| | | | CGATTTAAC |
| NRAS- | SNV | 106 | ATACTGGATACAGCTGGAAAAGAAGAGTAC |
| Q61K | WT | 103 | ATACTGGATACAGCTGGACAAGAAGAGTAC |
| (c.181C > | P | 107 | AAGGACGAGCAAATGTACCTGCAACTGGATACAGCTGGA |
| A) | | | A |
| | C | 108 | ACTCTTCTTTTCCAGCTGTATCCAGTTGGTCTACTATCCAC |
| | | | GATTTAAC |
| NRAS- | SNV | 109 | GGACATACTGGATACAGCTGGACTAGAAGA |
| Q61L | WT | 110 | GGACATACTGGATACAGCTGGACAAGAAGA |
| (c.182A > | P | 111 | AAGGACGAGCAAATGTACCTGCAACATACTGGATACAGC |
| T) | | | T |
| | C | 112 | TTCTAGTCCAGCTGTATCCAGTATGTTGGTCTACTATCCA |
| | | | CGATTTAAC |
| NRAS- | SNV | 113 | ATACTGGATACAGCTGGACGAGAAGAGTAC |
| Q61R | WT | 103 | ATACTGGATACAGCTGGACAAGAAGAGTAC |
| (c.182A > | P | 114 | AAGGACGAGCAAATGTACCTGCAACTGGATACAGCTGGA |
| G) | | | CG |
| | C | 115 | TACTCTTCTCGTCCAGCTGTATCCAGTTGGTCTACTATCCA |
| | | | CGATTTAAC |

TABLE 8
Sequences of SNV, WT, and target-dependent X-Probe strands for
PIK3CA mutations.

| | | SEQ | |
|---|---|---|---|
| | | ID | |
| Mutation | Species | NO. | Sequence |
| PIK3CA- | SNV | 116 | CTCTCTAAAATCACTGAGCAGGAGAAAGAT |
| E542K | WT | 117 | CTCTCTGAAATCACTGAGCAGGAGAAAGAT |
| (c.1624 | P | 118 | AAGGACGAGCAAATGTACCTGCACTCTAAAATCACTGAG |
| G > A) | | | CA |
| | C | 119 | TCTTTCTCCTGCTCAGTGATTTTAGAGTGGTCTACTATCCA |
| | | | CGATTTAAC |
| PIK3CA- | SNV | 120 | AGATCCTCTCTCTGAAATCACTAAGCAGGA |
| E545K | WT | 121 | AGATCCTCTCTCTGAAATCACTGAGCAGGA |
| (c.1633 | P | 122 | AAGGACGAGCAAATGTACCTGCAATCCTCTCTCTGAAATC |
| G > A) | | | AC |
| | C | 123 | CCTGCTTAGTGATTTCAGAGAGAGGATTGGTCTACTATCC |
| | | | ACGATTTAAC |
| PIK3CA- | SNV | 124 | TGATGCACTTCATGGTGGCTGGACAACAAA |
| H1047L | WT | 125 | TGATGCACATCATGGTGGCTGGACAACAAA |
| (c.3140 | P | 126 | AAGGACGAGCAAATGTACCTGCAATGCACTTCATGGTGG |
| A > T) | | | CT |
| | C | 127 | TGTTGTCCAGCCACCATGAAGTGCATTGGTCTACTATCCA |
| | | | CGATTTAAC |
| PIK3CA- | SNV | 128 | TGATGCACGTCATGGTGGCTGGACAACAAA |
| H1047R | WT | 125 | TGATGCACATCATGGTGGCTGGACAACAAA |
| (c.3140 | P | 129 | AAGGACGAGCAAATGTACCTGCAATGCACGTCATGGTGG |
| A > G) | | | CT |
| | C | 130 | TGTTGTCCAGCCACCATGACGTGCATTGGTCTACTATCCA |
| | | | CGATTTAAC |

TABLE 9
Sequences of SNV, WT, and target-dependent X-Probe strands for
STK11 mutations.

| | | SEQ | |
|---|---|---|---|
| | | ID | |
| Mutation | Species | NO. | Sequence |
| STK11- | SNV | 131 | ATCGACTCCACCGAGGTCATCTACTAGCCG |
| Q37* (c. | WT | 132 | ATCGACTCCACCGAGGTCATCTACCAGCCG |
| 109C > T) | P | 133 | AAGGACGAGCAAATGTACCTGCACGACTCCACCGAGGTC |
| | | | AT |
| | C | 134 | GCTGGTAGATGACCTCGGTGGAGTCGTGGTCTACTATCCA |
| | | | CGATTTAAC |
| STK11- | SNV | 135 | ATCCCGGGCGACTGTGGCCCCCTGCTCTCT |
| P281L | WT | 136 | ATCCCGGGCGACTGTGGCCCCCCGCTCTCT |
| (c.842 | P | 137 | AAGGACGAGCAAATGTACCTGCACCCGGGCGACTGTGGC |
| C > T) | | | CCC |
| | C | 138 | AGAGCAGGGGGCCACAGTCGCCCGGGTGGTCTACTATCC |
| | | | ACGATTTAAC |
| STK11- | SNV | 139 | AGGACCTCTTGGACATCGAGGATGACATCA |
| F354L | WT | 140 | AGGACCTCTTCGACATCGAGGATGACATCA |
| (c.1062 | P | 141 | AAGGACGAGCAAATGTACCTGCAGACCTCTTGGACATCG |
| C > G) | | | AG |
| | C | 142 | ATGTCATCCTCGATGTCCAAGAGGTCTGGTCTACTATCCA |
| | | | CGATTTAAC |

TABLE 10
Sequences of SNV, WT, and target-dependent X-Probe strands for
TP53 mutations.

| | | SEQ | |
|---|---|---|---|
| | | ID | |
| Mutation | Species | NO. | Sequence |
| TP53- | SNV | 143 | GTTGTGAGGCGCTGCCCCCACCATGAGCGC |
| R175H | WT | 144 | GTTGTGAGGCACTGCCCCCACCATGAGCGC |
| (c.524G > | P | 145 | AAGGACGAGCAAATGTACCTGCATGTGAGGCACTGCCCC |
| A) | | | CAC |
| | C | 146 | GCTCATGGTGGGGGCAGTGCCTCACATGGTCTACTATCCA |
| | | | CGATTTAAC |
| TP53- | SNV | 147 | ACTTTTTGACATAGTGTGGTGGTGCCCTAT |
| R213*(c. | WT | 148 | ACTTTTCGACATAGTGTGGTGGTGCCCTAT |
| 637C > T) | P | 149 | AAGGACGAGCAAATGTACCTGCATTTTTGACATAGTGTG |
| | | | GTG |
| | C | 150 | AGGGCACCACCACACTATGTCAAAAATGGTCTACTATCC |
| | | | ACGATTTAAC |
| TP53- | SNV | 151 | CGACATAGTGTGGTGGTGCCCTGTGAGCCG |
| Y220C | WT | 152 | CGACATAGTGTGGTGGTGCCCTATGAGCCG |
| (c.659A > | P | 153 | AAGGACGAGCAAATGTACCTGCAACATAGTGTGGTGGTG |
| G) | | | CCC |
| | C | 154 | GCTCACAGGGCACCACCACACTATGTTGGTCTACTATCCA |
| | | | CGATTTAAC |
| TP53- | SNV | 155 | TTCCTGCATGGGCGGCATGAACCAGAGGCC |
| R248Q | WT | 156 | TTCCTGCATGGGCGGCATGAACCGGAGGCC |
| (c.743G > | P | 157 | AAGGACGAGCAAATGTACCTGCACCTGCATGGGCGGCAT |
| A) | | | GA |
| | C | 158 | CCTCTGGTTCATGCCGCCCATGCAGGTGGTCTACTATCCA |
| | | | CGATTTAAC |
| TP53- | SNV | 159 | ATGAACTGGAGGCCCATCCTCACCATCATC |
| R248W | WT | 160 | ATGAACCGGAGGCCCATCCTCACCATCATC |
| (c.742C > | P | 161 | AAGGACGAGCAAATGTACCTGCAGAACTGGAGGCCCATC |
| T) | | | CT |
| | C | 162 | TGATGGTGAGGATGGGCCTCCAGTTCTGGTCTACTATCCA |
| | | | CGATTTAAC |
| TP53- | SNV | 17 | ACGGAACAGCTTTGAGGTGTGTGTTTGTGC |
| R273C | WT | 18 | ACGGAACAGCTTTGAGGTGCGTGTTTGTGC |
| (c.817C > | P | 15 | AAGGACGAGCAAATGTACCTGCAGGAACAGCTTTGAGGT |
| T) | | | GT |
| | C | 16 | ACAAACACACACCTCAAAGCTGTTCCTGGTCTACTATCCA |
| | | | CGATTTAAC |
| TP53- | SNV | 163 | AGGTGCATGTTTGTGCCTGTCCTGGGAGAG |
| R273H | WT | 164 | AGGTGCGTGTTTGTGCCTGTCCTGGGAGAG |
| (c.818G > | P | 165 | AAGGACGAGCAAATGTACCTGCAGTGCATGTTTGTGCCT |
| A) | | | GT |
| | C | 166 | CTCCCAGGACAGGCACAAACATGCACTGGTCTACTATCC |
| | | | ACGATTTAAC |
| TP53- | SNV | 167 | GGGAGAGACTGGCGCACAGAGGAAGAGAAT |
| R282W | WT | 168 | GGGAGAGACCGGCGCACAGAGGAAGAGAAT |
| (c.844C > | P | 169 | AAGGACGAGCAAATGTACCTGCAGAGAGACTGGCGCACA |
| T) | | | GA |
| | C | 170 | TCTCTTCCTCTGTGCGCCAGTCTCTCTGGTCTACTATCCAC |
| | | | GATTTAAC |

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

It is contemplated that any instance discussed in this specification can be implemented with respect to any method or composition of the invention, and vice versa. Furthermore, compositions of the invention can be used to achieve the methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

Each probe system described herein may be comprised of DNA, RNA, or analogs thereof, and/or combinations thereof. In certain instances, a probe system comprises one or more non-natural nucleotides. The incorporation of non-natural nucleotides in the primers can further augment the performance of the probe systems, such as by providing improved per-base binding affinity and increased nuclease resistance.

A “target” for a probe system described herein can be any single-stranded nucleic acid, such as single-stranded DNA and single-stranded RNA, including double-stranded DNA and RNA rendered single-stranded through heat shock, asymmetric amplification, competitive binding, and other methods standard to the art. A “target” for a primer system can be any single-stranded (ss) or double-stranded (ds) nucleic acid, for example, DNA, RNA, or the DNA product of RNA subjected to reverse transcription. In some instances, a target may be a mixture (chimera) of DNA and RNA. In other instances, a target comprises artificial nucleic acid analogs, for example, peptide nucleic acids (Nielsen et al. Science 254(5037): 1497-500 (1991)) or locked nucleic acids (Alexei et al. Tetrahedron 54(14): 3607-30 (1998)). In some instances, a target may be naturally occurring (e.g., genomic DNA) or it may be synthetic (e.g., from a genomic library). As used herein, a “naturally occurring” nucleic acid sequence is a sequence that is present in nucleic acid molecules of organisms or viruses that exist in nature in the absence of human intervention. In some instances, a target is genomic DNA, messenger RNA, ribosomal RNA, micro-RNA, pre-micro-RNA, pro-micro-RNA, long non-coding RNA, small RNA, epigenetically modified DNA, epigenetically modified RNA, viral DNA, viral RNA or piwi-RNA. In certain instances, a target nucleic acid is a nucleic acid that naturally occurs in an organism or virus. In some instances, a target nucleic is the nucleic acid of a pathogenic organism or virus. In certain instances the presence or absence of a target nucleic acid in a subject is indicative that the subject has a disease or disorder or is predisposed to acquire a disease or disorder. In certain instances the presence or absence of a target nucleic acid in a subject is indicative that the subject will respond well or poorly to a treatment, such as a drug, to treat a disease or disorder. In certain instances the presence or absence of a target nucleic acid in a subject is indicative that the subject who has been treated previously for cancer and is in remission may be at risk of cancer recurrence.

The terms “polynucleotide,” “nucleic acid,” “strand,” “oligonucleotide,” and “nucleic acid molecule” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. The term “recombinant” polynucleotide means a polynucleotide of genomic, cDNA, semi-synthetic, or synthetic origin which either does not occur in nature or is linked to another polynucleotide in a non-natural arrangement. The term “isolated nucleic acid” refers to a polynucleotide of natural or synthetic origin or some combination thereof, which (1) is not associated with the cell in which the “isolated nucleic acid” is found in nature, and/or (2) is operably linked to a polynucleotide to which it is not linked in nature.

A nucleic acid may also encompass single- and double-stranded DNA and RNA, as well as any and all forms of alternative nucleic acid containing modified bases, sugars, and backbones. The term “nucleic acid” thus will be understood to include, but not be limited to, single- or double-stranded DNA or RNA (and forms thereof that can be partially single-stranded or partially double-stranded), cDNA, aptamers, peptide nucleic acids (“PNA”), 2′-5′ DNA (a synthetic material with a shortened backbone that has a base-spacing that matches the A conformation of DNA; 2′-5′ DNA will not normally hybridize with DNA in the B form, but it will hybridize readily with RNA), and locked nucleic acids (“LNA”). Nucleic acid analogues include known analogues of natural nucleotides that have similar or improved binding, hybridization of base-pairing properties. “Analogous” forms of purines and pyrimidines are well known in the art, and include, but are not limited to aziridinylcytosine, 4-acetylcytosine, 5-fluorouracil, 5-bromouracil, 5-carboxymethylaminomethyl-2-thiouracil, 5-carboxymethylaminomethyluracil, inosine, N6-isopentenyladenine, 1-methyladenine, 1-Methylpseudouracil, 1-methylguanine, 1-methylinosine, 2,2-dimethylguanine, 2-methyladenine, 2-methylguanine, 3-methylcytosine, 5-methylcytosine, N.sup.6-methyladenine, 7-methylguanine, 5-methylaminomethyluracil, 5-methoxyaminomethyl-2-thiouracil, beta-D-mannosylqueosine, 5-methoxyuracil, 2-methylthio-N6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, and 2,6-diaminopurine. DNA backbone analogues provided herein include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs), methylphosphonate linkages or alternating methylphosphonate and phosphodiester linkages (Strauss-Soukup, 1997, Biochemistry 36:8692-8698), and benzylphosphonate linkages, as discussed in U.S. Pat. No. 6,664,057; see also OLIGONUCLEOTIDES AND ANALOGUES, A PRACTICAL APPROACH, edited by F. Eckstein, IRL Press at Oxford University Press (1991); Antisense Strategies, Annals of the New York Academy of Sciences, Volume 600, Eds. Baserga and Denhardt (NYAS 1992); Milligan, 1993, J. Med. Chem. 36: 1923-1937; Antisense Research and Applications (1993, CRC Press). The nucleic acids herein can be extracted from cells or synthetically prepared according to any means known to those skilled in the art; for example, the nucleic acids can be chemically synthesized or transcribed or reverse transcribed from cDNA or mRNA, among other sources.

A target nucleic acid utilized herein can be any nucleic acid, for example, human nucleic acids, bacterial nucleic acids, or viral nucleic acids. A target nucleic acid sample or sample comprising a target nucleic acid can be, for example, a nucleic acid sample from one or more cells, tissues, or bodily fluids. Target samples can be derived from any source including, but not limited to, eukaryotes, plants, animals, vertebrates, fish, mammals, humans, non-humans, bacteria, microbes, viruses, biological sources, serum, plasma, blood, urine, semen, lymphatic fluid, cerebrospinal fluid, amniotic fluid, biopsies, needle aspiration biopsies, cancers, tumors, tissues, cells, cell lysates, crude cell lysates, tissue lysates, tissue culture cells, buccal swabs, mouthwashes, stool, mummified tissue, forensic sources, autopsies, archeological sources, infections, nosocomial infections, production sources, drug preparations, biological molecule productions, protein preparations, lipid preparations, carbohydrate preparations, inanimate objects, air, soil, sap, metal, fossils, excavated materials, and/or other terrestrial or extra-terrestrial materials and sources. The sample may also contain mixtures of material from one source or different sources. For example, nucleic acids of an infecting bacterium or virus can be amplified along with human nucleic acids when nucleic acids from such infected cells or tissues are amplified using the disclosed methods. Types of useful target samples include eukaryotic samples, plant samples, animal samples, vertebrate samples, fish samples, mammalian samples, human samples, non-human samples, bacterial samples, microbial samples, viral samples, biological samples, serum samples, plasma samples, blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, crude cell lysate samples, tissue lysate samples, tissue culture cell samples, buccal swab samples, mouthwash samples, stool samples, mummified tissue samples, autopsy samples, archeological samples, infection samples, nosocomial infection samples, production samples, drug preparation samples, biological molecule production samples, protein preparation samples, lipid preparation samples, carbohydrate preparation samples, inanimate object samples, air samples, soil samples, sap samples, metal samples, fossil samples, excavated material samples, and/or other terrestrial or extra-terrestrial samples. In some instances, a target nucleic acids utilized herein comprise repetitive sequence, secondary structure, and/or a high G/C content.

In certain instances, a target nucleic acid molecule of interest is about 10 to about 1,000,000 nucleotides (nt) in length. In some instances, the target is about 19 to about 100, about 100 to about 1000, about 1000 to about 10,000, about 10,000 to about 100,000, or about 100,000 to about 1,000,000 nucleotides in length. In some instances, the target is about 20, about 100, about 200, about 300, about 400, about 500, about 600, about 700, about 800, about 900, about 1,000, about 2,000, about 3,000, about 4,000, about 5,000, about 6,000, about 7,000, about 8,000, about 9000, about 10,000, about 20,000, about 30,000, about 40,000, about 50,000, about 60,000, about 70,000, about 80,000, about 90,000, about 100,000, about 200,000, about 300,000, about 400,000, about 500,000, about 600,000, about 700,000, about 800,000, about 900,000, or about 1,000,000 nucleotides in length. It is to be understood that the target nucleic acid may be provided in the context of a longer nucleic acid (e.g., such as a coding sequence or gene within a chromosome or a chromosome fragment).

In certain instances, a target of interest is linear, while in other instances, a target is circular (e.g., plasmid DNA, mitochondrial DNA, or plastid DNA).

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A nucleic acid probe for hybridizing to a target nucleic acid in a sample comprising a first subsequence, a second subsequence, a third subsequence, a fourth subsequence, a fifth subsequence, a sixth subsequence, and a seventh subsequence, wherein the fourth and fifth subsequences are complementary to a ninth subsequence and an eighth subsequence of the target nucleic acid, respectively, and wherein the subsequences are arranged in oligonucleotides in the probe as follows:
a universal component comprising a first universal oligonucleotide and a second universal oligonucleotide, wherein the first universal oligonucleotide comprises the seventh subsequence, and wherein the second universal oligonucleotide comprises the first subsequence, and wherein the sequence of the first universal oligonucleotide and the second universal oligonucleotide including the seventh subsequence and first subsequence, is not complementary to a sequence of the target nucleic acid; and
a target-dependent component comprising a protector oligonucleotide and a complement oligonucleotide, wherein the protector oligonucleotide comprises the second subsequence and the third subsequence, wherein the complement oligonucleotide comprises the fourth subsequence, the fifth subsequence, and the sixth subsequence, wherein the first subsequence is complementary to the second subsequence, wherein the seventh subsequence is complementary to the sixth subsequence, and wherein the third subsequence is complementary to the fifth subsequence.

2. The nucleic acid probe of claim 1 comprising:
a first double-stranded region comprising the first subsequence and the second subsequence;
a second double-stranded region comprising the seventh subsequence and the sixth subsequence; and
a third double-stranded region comprising the third subsequence and the fifth subsequence.

3. The nucleic acid probe of claim 1 further comprising a label conjugated to the first universal oligonucleotide or the second universal oligonucleotide.

4. The nucleic acid probe of claim 3 further comprising a moiety conjugated to either the second universal oligonucleotide to the extent the first universal nucleotide comprises the label or to the first universal oligonucleotide to the extent the second universal oligonucleotide comprises the label, wherein the moiety is sufficient to quench expression of the label.

5. The nucleic acid probe of claim 1 wherein the second universal oligonucleotide further comprises a tenth subsequence and the first universal oligonucleotide further comprises an eleventh subsequence, wherein the tenth subsequence is complementary to the eleventh subsequence.

6. The nucleic acid probe of claim 5 comprising:
a first double-stranded region comprising the first subsequence and the second subsequence;
a second double-stranded region comprising the seventh subsequence and the sixth subsequence;
a third double-stranded region comprising the third subsequence and the fifth subsequence; and
a fourth double-stranded region comprising the tenth subsequence and the eleventh subsequence.

7. The nucleic acid probe of claim 5 further comprising a label conjugated to the eleventh subsequence of the first universal oligonucleotide.

8. The nucleic acid probe of claim 7 further comprising a moiety conjugated to the tenth subsequence of the second universal oligonucleotide, wherein the moiety is sufficient to quench expression of the label.

9. The nucleic acid probe of claim 5 further comprising a label conjugated to the tenth subsequence of the second universal oligonucleotide.

10. The nucleic acid probe of claim 5 further comprising a label conjugated to either the first universal oligonucleotide or the second oligonucleotide, wherein the third, fourth, fifth, tenth and eleventh subsequences each possess a nucleotide sequence that contributes to a standard free energy of hybridization (ΔG°_{r×n}) with the target nucleic acid of −4 kcal/mol to +4 kcal/mol as determined by the following equation: ΔG°_{r×n}=ΔG°₉₋₄−ΔG°₁₀₋₁₁−ΔG°_{ML}+(ΔG°₈₋₅−ΔG°₃₋₅)−ΔG°_{label}, wherein ΔG°₉₋₄ is the standard free energy of the hybridization between the ninth subsequence and the fourth subsequence, ΔG°₁₀₋₁₁ is the standard free energy of the hybridization between the tenth subsequence and eleventh subsequence, wherein ΔG°₈₋₅ is the standard free energy of the hybridization between the eighth subsequence and the fifth subsequence, wherein ΔG°₃₋₅ is the standard free energy of the hybridization between the third subsequence and the fifth subsequence, wherein ΔG°_{ML} is the standard free energy of the multi-loop formed at the junction of different hybridized subsequences, and ΔG°_{label} is the standard free energy difference between the thermodynamic contribution of the label either on the first universal oligonucleotide when it is in close proximity with the second universal oligonucleotide versus when they are delocalized or on the second universal oligonucleotide when it is in close proximity with the first universal oligonucleotide versus when they are delocalized.

11. The nucleic acid probe of claim 5 in a solution wherein the second universal oligonucleotide and the protector oligonucleotide are present in combination to provide a first concentration, wherein the first universal oligonucleotide, second universal oligonucleotide, protector oligonucleotide, and complement oligonucleotide are present in combination to provide a second concentration, wherein the first universal oligonucleotide or the second universal oligonucleotide have a label conjugated thereto, wherein the third, fourth, fifth, tenth and eleventh subsequences each possess a nucleotide sequence that contributes to a standard free energy of hybridization (ΔG°_{r×n}) with the target nucleic acid that is not between −4 kcal/mol to +4 kcal/mol as determined by the following equation: ΔG°_{r×n}=ΔG°₉₋₄−ΔG°₁₀₋₁₁−ΔG°_{ML}+(ΔG°₈₋₅−ΔG°₃₋₅)−ΔG°_{label}, wherein ΔG°₉₋₄ is the standard free energy of the hybridization between the ninth subsequence and the fourth subsequence, ΔG°₁₀₋₁₁ is the standard free energy of the hybridization between the tenth subsequence and eleventh subsequence, wherein ΔG°₈₋₅ is the standard free energy of the hybridization between the eighth subsequence and the fifth subsequence, wherein ΔG°₃₋₅ is the standard free energy of the hybridization between the third subsequence and the fifth subsequence, wherein ΔG°_{ML} is the standard free energy of the multi-loop formed at the junction of different hybridized subsequences, and ΔG°_{label} is the standard free energy difference between the thermodynamic contribution of the label either on the first universal oligonucleotide when it is in close proximity with the second universal oligonucleotide versus when they are delocalized or on the second universal oligonucleotide when it is in close proximity with the first universal oligonucleotide versus when they are delocalized, but wherein the standard free energy of hybridization with the target nucleic acid as determined by ΔG°_{r×n}=ΔG°₉₋₄−ΔG°₁₀₋₁₁−ΔG°_{ML}+(ΔG°₈₋₅−ΔG°₃₋₅)−ΔG°_{label} is within 5 kcal/mol of the standard free energy determined by −Rτ ln([BP]/[BPCA]), wherein R is the ideal gas constant, τ is temperature in Kelvin, [BP] is the first concentration, and [BPCA] is the second concentration.

12. The nucleic acid probe of claim 5 further comprising a twelfth subsequence and a thirteenth subsequence, wherein the twelfth subsequence is between the second and third subsequence in the protector oligonucleotide and the thirteenth subsequence is between the fifth and sixth subsequence in the complement oligonucleotide, and wherein the twelfth subsequence is complimentary to the thirteenth subsequence.

13. The nucleic acid probe of claim 12 comprising:
a first double-stranded region comprising the first subsequence and the second subsequence;
a second double-stranded region comprising the seventh subsequence and the sixth subsequence;
a third double-stranded region comprising the third subsequence and the fifth subsequence, and the twelfth subsequence and the thirteenth subsequence; and
a fourth double-stranded region comprising the tenth subsequence and the eleventh subsequence.

14. The nucleic acid probe of claim 12 further comprising a label conjugated to the eleventh subsequence of the first universal oligonucleotide.

15. The nucleic acid probe of claim 14 further comprising a moiety conjugated to the tenth subsequence of the second universal oligonucleotide, wherein the moiety is sufficient to quench expression of the label.

16. The nucleic acid probe of claim 12 further comprising a label conjugated to the tenth subsequence of the second universal oligonucleotide.

17. The nucleic acid probe of claim 12 further comprising a label conjugated to either the first universal oligonucleotide or the second oligonucleotide, wherein the third, fourth, fifth, tenth, eleventh, twelfth and thirteenth subsequences each possess a nucleotide sequence that contributes to a standard free energy of hybridization (ΔG°_{r×n}) with the target nucleic acid of −4 kcal/mol to +4 kcal/mol as determined by the following equation: ΔG°_{r×n}=ΔG°₉₋₄−ΔG°₁₀₋₁₁−ΔG°₁₂₋₁₃−ΔG°_{ML}+(ΔG°₈₋₅−ΔG°₃₋₅)−ΔG°_{label}, wherein ΔG°₉₋₄ is the standard free energy of the hybridization between the ninth subsequence and the fourth subsequence, ΔG°₁₀₋₁₁ is the standard free energy of the hybridization between the tenth subsequence and eleventh subsequence, wherein ΔG°₁₂₋₁₃ is the standard free energy of the hybridization between the twelfth subsequence and thirteenth subsequence, wherein ΔG°₈₋₅ is the standard free energy of the hybridization between the eighth subsequence and the fifth subsequence, wherein ΔG°₃₋₅ is the standard free energy of the hybridization between the third subsequence and the fifth subsequence, wherein ΔG°_{ML} is the standard free energy of the multi-loop formed at the junction of different hybridized subsequences, and ΔG°_{label} is the standard free energy difference between the thermodynamic contribution of the label either on the first universal oligonucleotide when it is in close proximity with the second universal oligonucleotide versus when they are delocalized or on the second universal oligonucleotide when it is in close proximity with the first universal oligonucleotide versus when they are delocalized.

18. The nucleic acid probe of claim 12 in a solution wherein the second universal oligonucleotide and the protector oligonucleotide are present in combination to provide a first concentration, wherein the first universal oligonucleotide, second universal oligonucleotide, protector oligonucleotide, and complement oligonucleotide are present in combination to provide a second concentration, wherein the first universal oligonucleotide or the second universal oligonucleotide have a label conjugated thereto, wherein the third, fourth, fifth, tenth, eleventh, twelfth and thirteenth subsequences each possess a nucleotide sequence that contributes to a standard free energy of hybridization (ΔG°_{r×n}) with the target nucleic acid that is not between −4 kcal/mol to +4 kcal/mol as determined by the following equation: ΔG°_{r×n}=ΔG°₉₋₄−ΔG°₁₀₋₁₁−ΔG°₁₂₋₁₃−ΔG°_{ML}+(ΔG°₈₋₅−ΔG°₃₋₅)−ΔG°_{label}, wherein ΔG°₉₋₄ is the standard free energy of the hybridization between the ninth subsequence and the fourth subsequence, ΔG°₁₀₋₁₁ is the standard free energy of the hybridization between the tenth subsequence and eleventh subsequence, wherein ΔG°₁₂₋₁₃ is the standard free energy of the hybridization between the twelfth subsequence and thirteenth subsequence, wherein ΔG°₈₋₅ is the standard free energy of the hybridization between the eighth subsequence and the fifth subsequence, wherein ΔG°₃₋₅ is the standard free energy of the hybridization between the third subsequence and the fifth subsequence, wherein ΔG°_{ML} is the standard free energy of the multi-loop formed at the junction of different hybridized subsequences, and ΔG°_{label} is the standard free energy difference between the thermodynamic contribution of the label either on the first universal oligonucleotide when it is in close proximity with the second universal oligonucleotide versus when they are delocalized or on the second universal oligonucleotide when it is in close proximity with the first universal oligonucleotide versus when they are delocalized, but wherein the standard free energy of hybridization with the target nucleic acid as determined by ΔG°_{r×n}=ΔG°₉₋₄−ΔG°₁₀₋₁₁−ΔG°₁₂₋₁₃−ΔG°_{ML}+(ΔG°₈₋₅−ΔG°₃₋₅)−ΔG°_{label} is within 5 kcal/mol of the standard free energy determined by −Rτ ln([BP]/[BPCA]), wherein R is the ideal gas constant, τ is temperature in Kelvin, [BP] is the first concentration, and [BPCA] is the second concentration.

19. The nucleic acid probe of claim 1 in a solution, wherein the solution comprises one or more additional protector oligonucleotides and complement oligonucleotides, wherein the third subsequence of the one or more additional protector oligonucleotides and the fourth, and fifth subsequences of the one or more additional complement oligonucleotides are modified to provide specificity to one or more additional target nucleic acids, and wherein the concentration of the first universal oligonucleotide and the second universal nucleotide in the solution is sufficient to accommodate the one or more additional protector and complement oligonucleotides.

20. The nucleic acid probe of claim 1 without the second universal oligonucleotide and without the second subsequence of the protector oligonucleotide and further comprising a label conjugated to the first universal oligonucleotide, or without the first universal oligonucleotide and without the sixth subsequence of the complement oligonucleotide and further comprising a label conjugated to the second universal oligonucleotide.
